(19) United States
(12) Patent Application Publication
(10) Pub. No.: US 2026/0007646 A1
(43) Pub. Date: Jan. 8, 2026

(54) METHODS FOR TREATING ACNE AND SKIN OILINESS WITH TAZAROTENE

(71) Applicant: BAUSCH HEALTH IRELAND LIMITED, DUBLIN (IE)

(72) Inventors: Eric GUENIN, Bridgewater, NJ (US); Tina LIN, Irvine, CA (US); Adarsh KONDA, Fremont, CA (US)

(21) Appl. No.: 19/111,745

(22) PCT Filed: Sep. 16, 2022
(86) PCT No.: PCT/IB2023/059208

§ 371 Date: Mar. 13, 2025

Related U.S. Application Data
(60) Provisional application No. 63/407,596, filed on Sep. 16, 2022.

(51) Int. Cl. A61K 31/4436; A61P 17/00; A61P 17/10

(52) U.S. Cl. CPC A61K 31/4436; A61P 17/00; A61P 17/10

(57) ABSTRACT

Methods are provided for treating acne vulgaris in subjects having acne vulgaris and oily skin and for treating truncal acne. Compositions comprising tazarotene or a pharmaceutically acceptable salt of tazarotenic acid in an oily-in-water emulsion are also provided for use in the methods for treating acne vulgaris in subjects having acne vulgaris and oils skin and in the methods for treating truncal acne.

### CROSS-REFERENCES TO RELATED APPLICATIONS

This application claims priority to and the benefit of U.S. Ser. No. 63/407,596, filed on Sep. 16, 2022, the entire disclosure of which is hereby incorporated by reference.

### BACKGROUND OF THE INVENTION

Oily skin is a frequent complaint of dermatology patients with or without acne [Endly DC, et al. J Clin Aesthet Dermatol. 2017; 10(8): 49-55 (hereinafter, “Endly, et al.”); Maia Campos PMBG, et al., Front Physiol. 2019; 10: 254 (hereinafter, “Maia Campos”]. Patients with oily skin may be concerned with excessive brightness/shininess, a “greasy” appearance, and enlarged pores, which can negatively affect quality of life [Endly, et al.; Maia Campos, et al.; Arbuckle R, et al., Health Qual Life Outcomes. 2008; 6:80; and Wu Y, et al., Int J Cosmet Sci. 2013; 35(5): 442-447]. Higher rates of facial sebum production may be associated with larger pore sizes [Roh M, Br J Dermatol. 2006; 155(5): 890-894]. Skin oiliness and pore size may differ by race, with some studies demonstrating larger amounts of sebum secretion or larger pore sizes in Black patients [Endly, et al., Pappas A, Dermatoendocrinol. 2013; 5(2): 319-324; and Rawlings AV, Int J Cosmet Sci. 2006; 28(2): 79-93] and others finding no difference [Grimes P, et al., Cutis. 2004; 73(6): 392-396; and Sugiyama-Nakagiri Y, et al., J Dermatol Sci. 2009; 53(2): 135-139.]. Sebum composition and production can also vary by age and gender, and in patients with or without acne [Youn S W, et al., Br J Dermatol. 2005; 153(5): 919-924; Pappas A, et al., Dermatoendocrinol. 2009; 1(3): 157-161; Luebberding S, et al., Int J Cosmet Sci. 2013; 35(5): 477-483; Camera E, et al., J Lipid Res. 2016; 57(6): 1051-1058; and Rahrovan S, Int J Womens Dermatol. 2018; 4(3): 122-130.].

Sebum plays an important role in skin health by protecting against moisture loss, providing antimicrobial activity, and delivering antioxidants to the skin surface [Makrantonaki E, et al., Dermatoendocrinol. 2011; 3(1): 41-49 (hereinafter, “Makrantonaki, et al.”); Sakuma T H, Skin Pharmacol Physiol. 2012; 25(5): 227-235.]. Excessive sebum production, however, interferes with follicular keratinization in the pilosebaceous unit and leads to pore blockage [Makrantonaki, et al.; and Moradi Tuchayi S, et al., Nat Rev Dis Primers. 2015; 1: 15029] Sebum composition may also be altered by abnormal proliferation of Cutibacterium acnes (C. acnes) which can induce production of proinflammatory mediators in sebocytes and cause inflammation [Makrantonaki, et al.; and Zouboulis C C, et al., J Eur Acad Dermatol Venereol. 2014; 28(5): 527-532]. Increased sebum production and inflammation, as well as follicular proliferation of C. acnes and abnormal keratinization are all factors implicated in the pathophysiology of acne vulgaris [Jappe U, Acta Derm Venereol. 2003; 83(4): 241-248; and Zaenglein, et al.].

Topical retinoids block several inflammatory pathways and promote normal desquamation [Makrantonaki, et al.; and Leyden J, et al., Dermatol Ther (Heidelb). 2017; 7(3): 293-304 (“Leyden, et al.”)], making them a mainstay of acne treatment [Zaenglein AL, et al., J Am Acad Dermatol. 2016; 74(5): 945-973.e933 (hereinafter, “Zaenglein, et al.”); and Leyden et al.]. Additionally, retinoid receptors are expressed in sebocytes, and topical retinoids may inhibit differentiation and lipid synthesis [Zouboulis C C, et al., Rev Endocr Metab Disord. 2016; 17(3): 319-334]. Retinoid use, however, can be limited by cutaneous irritation. Vehicles formulated with emollients/moisturizers may reduce this irritation [Leyden et al.] and naturally oily skin may also provide a protective effect. The topical retinoid tazarotene 0.1% cream has been shown to reduce apparent facial pore size [Kang S, et al., J Am Acad Dermatol. 2005; 52(2): 268-274], though whether it has a sebum suppressing effect is unknown [Endly, et al.]. A lower-dose 0.045% tazarotene polymeric lotion also demonstrated efficacy in reducing acne lesions with good tolerability and safety profiles in data from two phase 3 clinical trials of patients with moderate-to-severe acne [Tanghetti EA, et al., J Drugs Dermatol. 2020; 19(3): 272-279 (hereinafter, “Tanghetti EA I”; and Tanghetti EA, et al., J Drugs Dermatol. 2020; 19(1): 70-77 (hereinafter, “Tanghetti EA II”)], as well as in patients with skin of color [Bhatia N, et al., J Drugs Dermatol. 2020; 19(7): 727-734]. Furthermore, the unique formulation of the tazarotene 0.045% polymeric emulsion lotion may minimize irritation by allowing for simultaneous delivery of emollients/humectants and lower drug concentrations of the active ingredient compared with other commercially available formulations [Tanghetti EA, et al., J Dermatolog Treat. 2019; 32(4): 391-398 (hereinafter, “Tanghetti EA III”)].

Despite the advances made in treating acne, there is still a population of patients that have both acne and oily skin that would benefit from a treatment that improves both acne and oily skin. The present invention satisfies this need.

### BRIEF SUMMARY OF THE INVENTION

In general, the present invention provides topical compositions and methods for treating acne vulgaris in a subject with acne vulgaris and oily skin. The present invention also provides methods for treating truncal acne in subjects in need thereof.

In one embodiment, the present invention provides a method of treating acne vulgaris in a subject with acne vulgaris and oily skin. In another embodiment, the present invention provides a method of reducing skin oiliness in a subject with acne vulgaris and oily skin. In another embodiment, the present invention provides methods for treating truncal acne is a subject in need thereof. It is noted that throughout this disclosure the term “acne vulgaris” is used interchangeably with the term “acne.”

In one aspect, the methods of the present invention involve administering a topical pharmaceutical composition that is useful for treating acne and reducing skin oiliness in a subject with acne and oily skin, the composition comprising: tazarotene or a pharmaceutically acceptable tazarotenic acid salt, wherein the tazarotene or the pharmaceutically acceptable tazarotenic acid salt is present in the composition at a positive concentration of less than 0.050 percent by weight of the composition; and a dermatologically acceptable oil-in-water emulsion vehicle.

In another aspect, this invention provides a method for treating acne in a subject with acne and oily skin, the method comprising topically applying a pharmaceutical composition to the skin of an affected area (e.g., the face) of the body of the subject having acne and oily skin, wherein the composition comprises: tazarotene or a pharmaceutically acceptable tazarotenic acid salt present in the composition at a positive concentration of less than 0.050 percent by weight of the composition; and a dermatologically acceptable oil-in-water emulsion vehicle.

In another aspect, this invention provides a method for improving (e.g., reducing) skin oiliness in a subject with acne and oily skin, the method comprising topically applying a pharmaceutical composition to the skin of an affected area of the body of the subject having acne and oily skin, wherein the composition comprises: tazarotene or a pharmaceutically acceptable tazarotenic acid salt present in the composition at a positive concentration of less than 0.050 percent by weight of the composition; and a dermatologically acceptable oil-in-water emulsion vehicle. In certain embodiments, the subject has moderate-to-severe acne.

In another aspect, this invention provides a method for treating truncal acne in a subject, the method comprising topically applying a pharmaceutical composition to an affected area of the trunk of the skin of an affected area of the body of the subject having acne and oily skin, wherein the composition comprises: tazarotene or a pharmaceutically acceptable tazarotenic acid salt present in the composition at a positive concentration of less than 0.050 percent by weight of the composition; and a dermatologically acceptable oil-in-water emulsion vehicle. In certain embodiments, the subject has moderate-to-severe acne. In one embodiment, the truncal acne is on the back or a portion of the back of the subject. In another embodiment, the truncal acne is on the chest or a portion of the chest of the subject. In another embodiment, the truncal acne is on one or both arms or a portion of one or both arms of the subject.

According to other aspects, the topical pharmaceutical composition may comprise tazarotene or the salt thereof at 0.01 to 0.049 percent by weight of the composition, or at about 0.045 percent by weight of the composition.

According to another aspect, the tazarotene or tazarotenic acid salt may be dissolved in a liquid oil component of the emulsion.

According to another aspect, the topical pharmaceutical composition may contain the tazarotene or the tazarotenic acid salt as a sole active pharmaceutical ingredient.

According to various aspects, the oil phase of the emulsion may comprise a liquid oil component comprising a DCAE, a MCAE, or combinations thereof. In other aspects, the oil phase includes diethyl sebacate as the DCAE.

According to other aspects, the aqueous phase of the emulsion may comprise water and a carbomer homopolymer.

According to another aspect, the topical pharmaceutical composition may comprise: an aqueous phase comprising water, a carbomer homopolymer, and a polymeric emulsifier; an oil phase comprising at least one member selected from the group consisting of a dicarboxylic acid ester and a mineral oil, and the tazarotene or the tazarotenic acid salt. According to other aspects, the aqueous phase may further comprise a humectant or a preservative, such as at least one member selected from the group consisting of methylparaben, propylparaben and sorbitol.

According to other aspects, the topical pharmaceutical composition may be in the form of a lotion or of a cream.

According to another aspect, the topical pharmaceutical composition may have a pH of about 4 to about 6.

For the methods of this invention, the step of applying may be carried out once per day for at least eight weeks. According to one aspect, the step of applying is carried out once per day for at least twelve weeks.

According to other aspects, the composition may be applied is to an affected area of a body, such as the face, of a subject suffering from acne vulgaris and oily skin in order both to treat the acne and oily skin.

Other features and advantages of the present invention will become apparent from the accompanying drawings and the following detailed description and claims.

### BRIEF DESCRIPTION OF THE DRAWINGS

FIG. 1 illustrates the oily skin assessment used to assess.

FIGS. 2A-2B illustrate reductions in acne lesion counts by visit in all participants with oily skin (ITT population, pooled). FIG. 2A illustrates the reduction in inflammatory acne lesions, and FIG. 2B illustrates the reductions in noninflammatory acne lesions.

FIG. 3 Illustrates improvements in oily skin from baseline to week 12 (ITT population, pooled). FIG. 3A illustrates improvements in oily skin for all participants, and FIG. 3B illustrates improvements in oily skin for Black participants.

FIGS. 4A-4C illustrate improvements with tazarotene 0.045% lotion in participants with oily skin. FIG. 4A illustrates improvements in oily skin for a 19-year-old Hispanic/American Indian or Alaska Native Male subject. FIG. 4B illustrates improvements in oily skin for a 19-year-old Non-Hispanic/Black male subject. FIG. 4C illustrates improvements in oily skin for a 18-year-old Non-Hispanic/White female subject.

FIGS. 5A-5B illustrate cutaneous safety and tolerability at baseline and week 12(safety population, pooled). FIG. 5A includes all participants with oily skin, and FIG. 5B includes Black participants with oily skin.

FIG. 6 illustrates the polymeric emulsion technology for the tazarotene 0.045% lotion.

FIG. 7 illustrates the efficacy of TAZ in truncal acne. The graph on the left illustrates clear/almost clear skin results, with greater than 90% of the participants achieving clear or almost clear skin at week 12. The graph on the right illustrates total lesion reduction, with the participants achieving greater than 80% reduction from baseline in total lesion counts at week 12.

FIG. 8 illustrates the cutaneous tolerability and safety, demonstrating that most participants had no tolerability issues. No significant changes from baseline to week 12 were observed in any tolerability assessment.

FIG. 9 illustrates results from the patient preference questionnaire.

FIG. 10 illustrates the mean spreadability of Tazarotene lotion and Trifaroteme cream, and the data obtained demonstrates that on average, skin coverage with 0.045% tazarotene lotion was about 30% greater than with trifarotene cream.

FIG. 11 is a graph of EGSS by visit.

FIG. 12 is a graph of change in inflammatory lesions from baseline.

FIG. 13 is a graph of change in non-inflammatory lesions from baseline.

FIGS. 14A-14C are graphs of cutaneous tolerability.

FIGS. 15A-15D include graphs of cutaneous safety.

### DETAILED DESCRIPTION OF THE INVENTION

In general, the present invention provides topical compositions and methods for treating acne vulgaris in a subject with acne vulgaris and oily skin. The present invention also provides methods for treating truncal acne in subjects in need thereof. The present invention provides a method of treating acne vulgaris in a subject with acne vulgaris and oily skin. The present invention also provides a method of reducing skin oiliness in a subject with acne vulgaris and oily skin. The present invention further methods for treating truncal acne is a subject in need thereof.

Throughout this disclosure, the term “acne vulgaris” is used interchangeably with the term “acne.” Moreover, throughout this disclosure, unless otherwise indicated, the concentration of an ingredient of the composition is in percent by weight of the total composition.

### A. Tazarotene Compositions

In one aspect, the topical pharmaceutical composition used in the methods of the present invention comprises tazarotene, or a pharmaceutically acceptable salt of tazarotenic acid, at a concentration below that which is often utilized in topical formulations. For example, tazarotene or the tazarotenic acid salt is included in a composition at a positive concentration less than 0.05 wt %.

In certain embodiments of the present invention, tazarotene or the tazarotenic acid salt is present in the composition at a positive concentration of less than 0.05% based on the weight of the composition (“wt %”). For example, this component is present in an amount ranging from about 0.01 to about 0.049 wt %, or from about 0.01 to about 0.045 wt %, or from about 0.02 to about 0.045 wt %, or from about 0.03 to about 0.045 wt %, or at about 0.045 wt %. Specific concentrations of this component include the following: 0.01 wt %, 0.015 wt %, 0.02 wt %, 0.025 wt %, 0.03 wt % 0.035 wt %, 0.04 wt %, and 0.045 wt %.

In one aspect, the topical pharmaceutical composition comprises an oil-in-water emulsion as a carrier vehicle, in which an internal oil phase is dispersed in a continuous aqueous phase. The emulsion may be a macroemulsion, a microemulsion, or a nanoemulsion. The composition may have the dosage form of gel, lotion, or cream, or ointment, or liquid, or oil, or spray or foam. In one embodiment, the composition is in the form of a lotion. In another embodiment, the composition is in the form of a cream.

In addition to the tazarotene active ingredient, the composition used in the methods of the present invention can comprise one or more dermatologically acceptable excipients, such as liquid oils, waxes viscosity-modifying agents, thickening agents, gelling agents, alcohols, surfactants, chelating agents, buffers, preservatives, humectants, emollients, stabilizers, diluents, dispersing agents, emulsifiers, wetting agents, stabilizers, pH adjusters, solvents or cosolvents.

The composition used in the methods of the present invention can include a thickening agent to provide the desired viscosity so that the composition can be provided in the form of a gel, lotion, cream, or ointment. The thickening agent can be miscible or soluble in an aqueous fluid. Non-limiting examples of suitable thickening agents include, but are not limited to, acacia, alginic acid and its salts, hyaluronic acid and its salts, carbomers (also known as carboxy vinyl polymers, which are cross-linked polyacrylic acid), carboxymethylcellulose, ethylcellulose, gelatin, hydroxyethyl cellulose, hydroxypropyl cellulose, hydroxypropylmethyl cellulose, methylcellulose, poloxamers, polyvinylpyrrolidone, polyvinyl alcohol, tragacanth, xanthan gum, magnesium aluminum silicate, and bentonite. The thickening agent can also reside in (or be incorporated into) the oil or lipophilic portion of the formulation. Examples of suitable lipophilic thickening agents include, but are not limited to, cetyl alcohol, stearyl alcohol, glyceryl stearate, white beeswax, microcrystalline wax, hydrogenated polyisobutane polymers, and emulsifying wax.

A suitable group of thickening agents is the carbomer group of thinking agents, including, for example, Carbopol® and polycarbophil (The Lubrizol Corporation, Wickliffe, Ohio). Carbopol® homopolymers are polymers of acrylic acid crosslinked with allyl sucrose or allylpentaerythritol. Carbopol® copolymers are polymers of acrylic acid and C10-C30 alkyl acrylate crosslinked with allylpentaerythritol. Carbopol® interpolymers are carbomer homopolymers or copolymers that contain a block copolymer of polyethylene glycol and a long chain alkyl acid ester. Noveon® polycarbophil is a polymer of acrylic acid crosslinked with divinyl glycol.

A surfactant or emulsifier is optionally included, if desired or required. Pharmaceutically acceptable anionic, cationic, or non-ionic surfactants can be included in a composition used in the methods of the present invention. Non-ionic surfactants are preferred. Non-limiting examples of non-ionic surfactants include, but are not limited to, Octoxynol (also known as Macrogol tetramethylbutylphenyl ether, octylphenoxy polyethoxyethanol, or polyoxyethylene octylphenyl ether), such as Octoxynol 1, 3, 5, 8, 9, 10, 12, 13, 16, 30, 40, 70 (wherein the number indicates the number of repeating oxyethylene units), or other Octoxynols that comprise different numbers of repeating units of oxyethylene in the side chain, sorbitan esters (such as sorbitan monooleate and sorbitan monostearate, commonly known by their trade names Span 80 and Span 60), polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F127 or Pluronic® F108), or poloxamines (synthetic block polymers of ethylene oxide and propylene oxide attached to ethylene diamine, such as those commonly known by their trade names of Tetronic®; e.g., Tetronic® 1508 or Tetronic® 908, etc.), other nonionic surfactants such as Brij® (polyoxyethylene alkyl ether having a formula of CH₃—(CH₂)₁₀₋₁₆—(O—C₂H₄)₁₋₂₅—OH), Myrj® (stearic acid esterified with polyoxyethylene having 40-100 repeating oxyethylene units), and long chain fatty alcohols (e.g., oleyl alcohol, stearyl alcohol, myristyl alcohol, docosahexaenoyl alcohol, etc.) with carbon chains having about 12 or more carbon atoms (e.g., such as from about 12 to about 24 carbon atoms).

In addition, polymeric emulsifiers such as those known under the trade name Pemulen™ (The Lubrizol Corporation, Wickliffe, Ohio) can be used. These are polymers of acrylic acid, modified by long chain (C₁₀-C₃₀) alkyl acrylates, and crosslinked with allylpentaerythritol. Examples of the Pemulen™ polymeric emulsifies include, for example, Pemulen™ TR-1 and Pemulen™ TR-2.

An anionic emulsifier may be used, such as sodium or potassium oleate, triethanolamine stearate, sodium lauryl sulfate, sodium dioctyl sulfosuccinate, and sodium docusate. Less preferred are cationic emulsifiers such as quaternary ammonium salts. Still other emulsifiers include glyceryl monostearate, polyoxyethylene monooleate, polyoxyethylene monostearate, polyoxyethylene monolaurate, potassium oleate, sodium lauryl sulfate, sodium oleate, sorbitan monolaurate, sorbitan monopalmitate, sorbitan monostearate, sorbitan tristearate, sorbitan monooleate, sorbitan trioleate, triethanolamine oleate, polyoxyethylene sorbitan monolaurate, polyoxyethylene sorbitan monopalmitate, polyoxyethylene sorbitan monostearate, polyoxyethylene sorbitan tristearate, polyoxyethylene sorbitan monooleate, and polyoxyethylene sorbitan trioleate.

The formulation desirably contains a dermatologically acceptable humectant including, but not limited to, glycerin, sorbitol, hexylene glycol, propylene glycol, or urea. In addition, the formulation may contain an emollient such as petrolatum, lanolin, mineral oil, light mineral oil, stearic acid, cyclomethicone, or dimethicone. Chelating agents such as EDTA and citric acid and their salts can be included in compositions used in the methods of the present invention.

The liquid oil component of the composition includes one or more materials that are practically insoluble or insoluble in water and which are liquid at room temperature. For example, in one embodiment, the liquid oil component of the composition includes one or more materials that are practically insoluble or insoluble in water and which are liquid at room temperature of 22° C. The liquid oil component can be selected from one or more ingredients including, but not limited to, dicarboxylic acid esters (“DCAE”), monocarboxylic acid esters (“MCAE”), fish-liver oil, long-chain triglycerides (wherein each side chain has 14-18 carbons, such as peanut oil, sesame oil, coconut oil, sunflower oil, corn oil, olive oil, cotton seed oil, or derivatives thereof), propylene glycol diesters, medium-chain triglycerides (such as those wherein each side chains has 8-10 carbons; e.g., capric/caprylic acid triglycerides), hydrocarbons like mineral oil, light mineral oil, squalene, and squalane, fatty alcohols (such as octyldodecanol and isostearyl alcohol), and fatty acids (such as isostearic acid and oleic acid).

In some embodiments, the liquid oil component comprises a dicarboxylic acid ester and light mineral oil. In some other embodiments, the liquid oil component comprises one or more long-chain triglycerides.

The compositions used in the methods of the present invention can include other lipophilic liquids in an amount that is sufficient to be miscible with the dicarboxylic acid ester and/or monocarboxylic acid ester. The lipophilic liquid may be an emollient such as lanolin oil, mineral oil, light mineral oil, isostearic acid, squalene, octyldodecanol, fractionated coconut oil, cyclomethicone, or dimethicone.

In addition to the liquid oil component, the formulation can contain water insoluble or practically insoluble ingredients that are not liquid at room temperature, but are soluble in the liquid oil component.

DCAEs suitable for use in the compositions used in the methods of the present invention have the formula R₁OOC—(CH₂)ₙ—COOR₂, wherein R₁ and R₂ are alkyl groups containing between 1 and 4 carbons or aryl groups and may be the same or may be different and wherein (CH₂)ₙ is a straight or branched chain and n is between 1 and 12. Examples of DCAEs containing one or more aryl groups are dibenzyl esters of dicarboxylic acids. A preferred dicarboxylic acid ester is diethyl sebacate, which has the formula CH₃CH₂OOC—(CH₂)₈—COOCH₂CH₃. Examples of other suitable dicarboxylic acid esters (where R₁ and R₂ are the same) are dimethyl, diethyl, dipropyl, diisopropyl, dibutyl and diisobutyl esters such as oxalate, malate, succinate, glutarate, adipate, pimelate, suberate, and azalate. Examples of suitable dicarboxylic acid esters (where R₁ is different from R₂) are methyl ethyl, methyl propyl, methyl butyl, methyl isopropyl, ethyl propyl, ethyl butyl, ethyl isopropyl, and propyl butyl esters such as oxalate, malate, succinate, glutarate, adipate, pimelate, suberate, azalate, and sebacate.

In some aspects, diethyl sebacate is included at 0.1 to 20 wt %, or at 0.5 to 10 wt %, or at 2 to 4 wt % of the weight of the composition.

Alternatively, or in combination with the DCAE, the composition can contain a MCAE. MCAEs suitable for use in the compositions used in the methods of the present invention have the formula CH₃—(CH₂)ₙ—COOR₁, wherein R₁ is an alkyl group containing between 1 and 4 carbons or an aryl group, and wherein (CH₂)ₙ is straight or branched chain and n is between 1 and 12. Examples of such monocarboxylic acid esters include methyl, ethyl, propyl, isopropyl, butyl, or an aryl such as benzyl formate, acetate, propionate, butyrate, valerate, laurate, myristate, palmitate, and stearate. Examples of preferred monocarboxylic acid esters are isopropyl palmitate and isopropyl myristate.

The liquid oil phase can advantageously be used to dissolve one or more of the active ingredients within the emulsion. In one embodiment, for example, the tazarotene component is dissolved in the liquid oil phase within the formulation at room temperature. In another embodiment the tazarotene component is suspended within the formulation at room temperature. In the case wherein the tazarotene component is suspended in the formulation, this suspended active ingredient may be micronized, namely that the mean particle size is preferably about 25 microns in diameter or less.

In one aspect, a composition of the present invention comprises the ingredients at the concentrations shown in Table 1.

TABLE 1
Compositions of the Present Invention for Treating Skin Diseases

Concentration (wt %)

| Ingredient | Range 1 | Range 2 | Range 3 |
|---|---|---|---|
| Tazarotene or | 0.01-0.049 | 0.02-0.045 | 0.03-0.045 |
| Tazarotenic Acid | | | |
| Compound | | | |
| Emollient, Solvent, | 0.5-40 | 1-25 | 2-20 |
| and/or Thickener | | | |
| Emulsifier | 0.25-10 | 0.5-7 | 1-5 |
| Humectant | 0-15 | 2-12 | 3-10 |
| Polymeric Thickener | 0.05-2 | 0.1-1.5 | 0.3-1 |
| Pharmaceutical Aids | q.s. | q.s. | q.s. |
| Purified water | q.s. to 100 | q.s. to 100 | q.s. to 100 |

Non-limiting examples of compositions of the present invention are shown in Table 2.

TABLE 2
Some Emulsion Compositions of the Present Invention for Treating Acne and Oily Skin

| | | Concentration (wt %) | | | |
|---|---|---|---|---|---|
| Ingredient | Function | Range 1 | Range 2 | Range 3 | Composition A |
| Tazarotene | Retinoid | 0.02-0.049 | 0.03-0.049 | 0.04-0.049 | 0.045 |
| Diethyl sebacate | Liquid Oil & | 1-5 | 2-4 | 2.5-3.5 | 2.97 |
| | Solvent | | | | |
| Light mineral oil | Liquid Oil & Co- | 5-15 | 5-10 | 7.5-8.5 | 8.03 |
| | Solvent | | | | |
| Sorbitan | Surfactant/ | 0.01-1 | 0.02-0.5 | 0.05-0.2 | 0.1 |
| monooleate | Emulsifying | | | | |
| | Agent | | | | |
| Sorbitol | Humectant | 5-15 | 7-12 | 10-11 | 10.7 |
| solution, 70% | | | | | |
| Methyl paraben | Antimicrobial | 0.05-0.3 | 0.1-0.3 | 0.1-0.2 | 0.17 |
| | Preservative⁽¹⁾ | | | | |
| Propyl paraben | Antimicrobial | 0.01-0.1 | 0.01-0.05 | 0.02-0.04 | 0.03 |
| | Preservative⁽¹⁾ | | | | |
| Edetate | Chelating | 0.02-0.1 | 0.02-0.7 | 0.03-0.06 | 0.05 |
| disodium | Agent⁽¹⁾ | | | | |
| dihydrate | | | | | |
| Carbomer | Emulsifying | 0.1-1 | 0.2-0.7 | 0.3-0.5 | 0.4 |
| copolymer type | Agent | | | | |
| B (e.g., | | | | | |
| Pemulen ™ TR-1) | | | | | |
| Carbomer | Thickener | 0.2-1.5 | 0.3-1 | 0.5-0.7 | 0.6 |
| homopolymer | | | | | |
| type A (e.g., | | | | | |
| Carbomer 981) | | | | | |
| Sodium | pH-adjusting | q.s. to pH | q.s. to pH | q.s. to pH | q.s. to pH |
| hydroxide, 10% | Agent⁽¹⁾ | of 5.5 ± 0.5 | of 5.5 ± 0.5 | of 5.5 ± 0.5 | of 5.5 ± 0.5 |
| Solution | | | | | |
| Purified water | Carrier | q.s. to 100 | q.s. to 100 | q.s. to 100 | q.s. to 100 |

Note:
⁽¹⁾These ingredients are broadly classified as Pharmaceutical Aids.

Exemplary compositions suitable for use in all of the methods disclosed herein are disclosed in U.S. Pat. No. 11,311,483, the teachings of which are incorporated herein by reference for all purposes.

In one embodiment, the composition used in the methods of the present invention comprises: tazarotene at a concentration of about 0.045 percent by weight of the composition as a sole active pharmaceutical ingredient; the composition is an oil-in-water emulsion lotion comprising an oil phase and an aqueous phase; the oil phase comprises a dicarboxylic acid ester and a mineral oil at a total concentration of 10-12 percent by weight of the composition; the aqueous phase comprises water and a carbomer homopolymer, wherein the concentration of the carbomer homopolymer is 0.5-0.7 percent by weight of the composition; and the composition further comprises a polymeric emulsifier at a concentration of 0.3-0.5 percent by weight of the composition. In one embodiment, the dicarboxylic acid ester in the oil phase is diethyl sebacate. In another embodiment, the oil phase comprises diethyl sebacate and light mineral oil. In one embodiment, the tazarotene is in solution in the oil phase of the oil-in-water emulsion lotion. In one embodiment, the composition has a pH of about 4 to about 6.

In one embodiment, the composition used in the methods of the present invention consists essentially of 0.045 weight % tazarotene; diethyl sebacate; light mineral oil; sorbitan monooleate; sorbitol solution, 70%; methylparaben; propylparaben; edetate disodium dihydrate; carbomer polymer type B; carbomer homopolymer type A; sodium hydroxide; purified water.

In one embodiment, the composition used in the methods of the present invention consists essentially of: 0.045 weight % tazarotene; 2.5-3.5 weight % diethyl sebacate; 7.5-8.5 weight % light mineral oil; 0.05-0.2 weight % sorbitan monooleate; 10-11 weight % sorbitol solution, 70%; 0.1-0.2 weight % methylparaben; 0.02-0.04 weight % propylparaben; 0.03-0.06 weight % edetate disodium dihydrate; 0.3-0.5 weight % carbomer polymer type B; 0.5-0.7 weight % carbomer homopolymer type A; sodium hydroxide, q.s. to pH 5.5±0.5; and purified water, q.s. to 100 weight %.

A lotion having a composition as shown Composition A of Table 2 may be prepared using the following method.

A separate aqueous phase is made. In a manufacturing vessel equipped with a mixing implement (such as a propeller) and temperature control, purified water and disodium edetate dihydrate are combined and the mixture is agitated until a clear solution is achieved. Sorbitol, methylparaben, and propylparaben are then added to the mixture. The mixture is continuously mixed and heated to approximately 75° C. The mixture is agitated until a solution is obtained. The mixture is then removed from the heat source and allowed to cool to below 40° C. with continued mixing. With continuous mixing, Carbopol® 981 and Pemulen™ TR-1 carbomers are added to the mixture and dispersed. Mixing continues until the two carbomers are fully dispersed and hydrated.

A separate oil phase is made. In a vessel equipped with a mixing implement such as a propeller, diethyl sebacate, and tazarotene are combined. The mixture is agitated until a solution is achieved. With continuous mixing, light mineral oil and sorbitan monooleate are added. Mixing is continued until a solution is obtained.

In a separate vessel, an approximate 2.5 N solution of sodium hydroxide is prepared.

With high speed mixing, the oil phase containing the active ingredient (tazarotene) is added to the aqueous phase. Mixing is continued until a homogeneous emulsion is obtained. Mixing speed is decreased and mixing continued for an additional time of 10 minutes to 1 hour. With continuous mixing, an appropriate amount of the sodium hydroxide solution is added incrementally to obtain a pH of 5.5±0.5. Mixing continues further until a homogeneous lotion is obtained, such as for 30 minutes to 3 hours.

### B. Methods of Treating Acne and Oily Skin

In one aspect, the present invention provides a method for treating acne in a patient with acne and oily skin. In another aspect, the present invention provides a method for treating acne in a subject with moderate-to-severe acne and oily skin. The methods comprises topically applying to an affected area of the body of a subject suffering from acne and oily skin any one of the compositions of the present invention, as disclosed herein, one or more times per day for a period of time sufficient to treat such acne and skin oiliness. For example, such a period of time may be 1 to 30 days or longer as needed. For example, such a period of time may be one week, two weeks, four weeks, eight weeks, twelve weeks, or longer as needed. For example, a composition of the present invention is applied topically to affected areas of the body once per day for 7-14 days. Alternatively, it may be applied two or three times per day for 7-14 days. Alternatively, it may be applied once per day for one week to six months. For example, it may be applied once per day for two weeks, four weeks, eight weeks, or twelve weeks. In one embodiment, the treatment may be stopped for 1-7 days (e.g., 2, 3, 4, 5, 6, or 7 days) after an extended treatment period before it is resumed for another extended treatment period. Such an extended period may be 7 days, 7-14 days, 7-21 days, 7-30 days, or longer before more treatment is needed or desired.

In yet another aspect, the present invention provides a method of treating acne topically in a subject with oily skin with any of the pharmaceutical compositions of this invention, wherein the composition is applied once daily for more than 2 weeks, such as 4 weeks, for 6 weeks, for 8 weeks or for 12 weeks, without any serious adverse events, and/or with an improved adverse event profile compared to 0.1% tazarotene cream.

In yet another aspect, the present invention provides a method of treating acne topically in a subject with oily skin with any of the pharmaceutical compositions of this invention, wherein the composition is applied once daily for 12 weeks, and has better clinical efficacy and a lower adverse event profile as compared to commercial compositions containing 0.1% tazarotene.

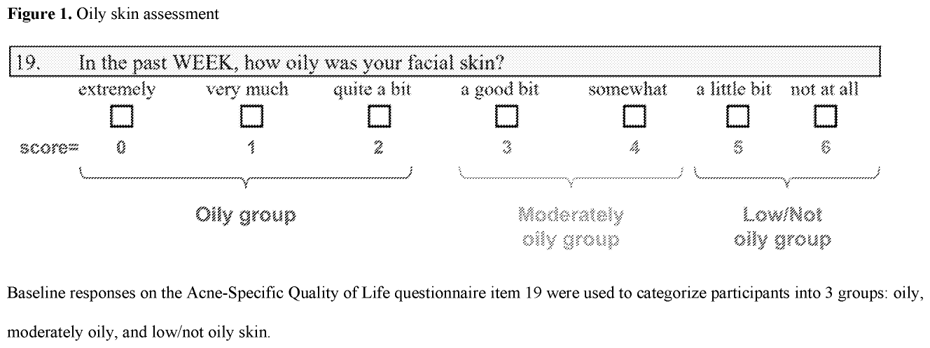

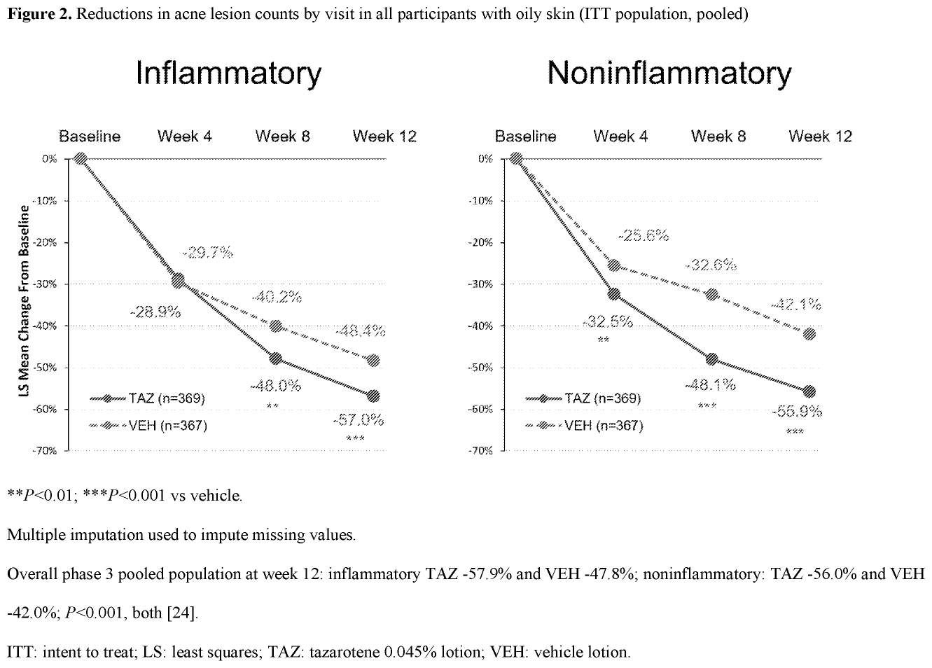

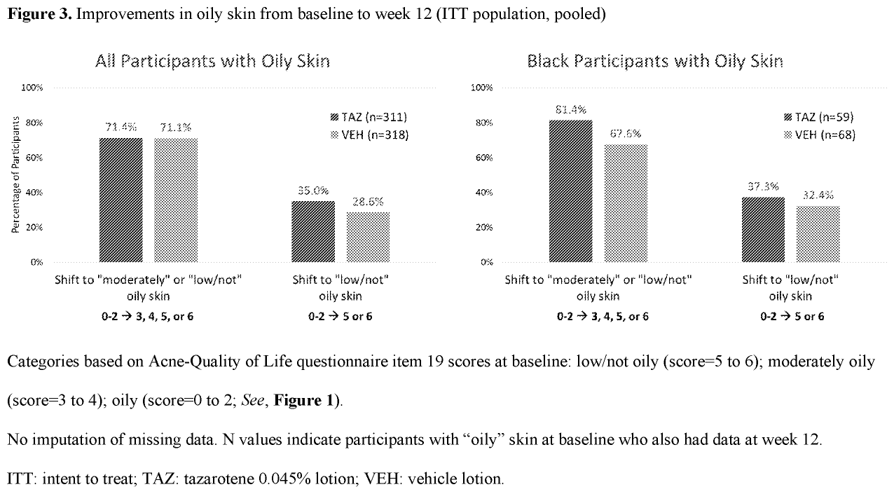

In the above methods of treating acne in a subject with acne and oily skin, the subject may be any person suffering from both acne and oily skin. In some embodiments, the oily skin assessment is made by the subject using a self-reporting scale similar to the one shown in FIG. 1. As shown in FIG. 1, a self-reported score of 0, 1, or 2 indicates oily skin. In other embodiments, the oily skin assessment or measurement can be made by a dermatologist or dermatological professional, such as a physician assistant, via physical observation of the skin to be treated or by using an oily skin assessment scale similar to the one shown in FIG. 1. An improvement or reduction in skin oiliness can also be assessed post-treatment by physical observation or by using an oily skin assessment scale similar to the one shown in FIG. 1. As will be appreciated by those of skill in the art, other oily assessments scales and methods can be used to assess the skin oiliness of a subject both prior to treatment and post-treatment.

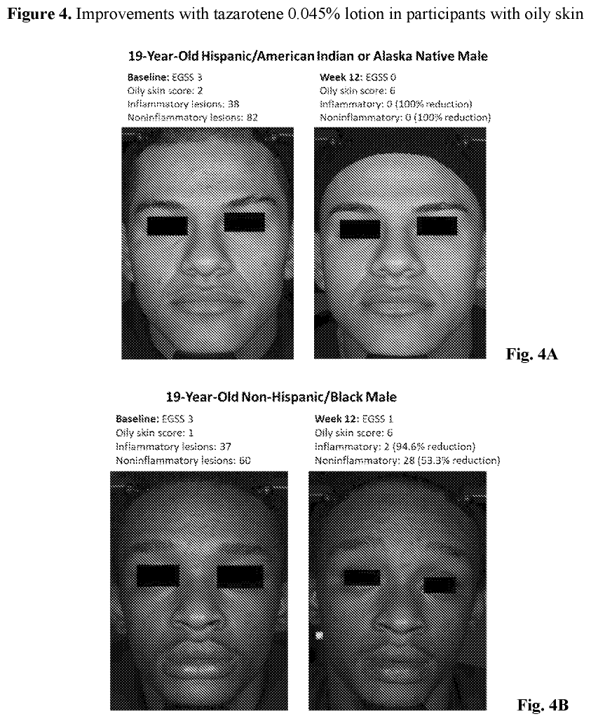

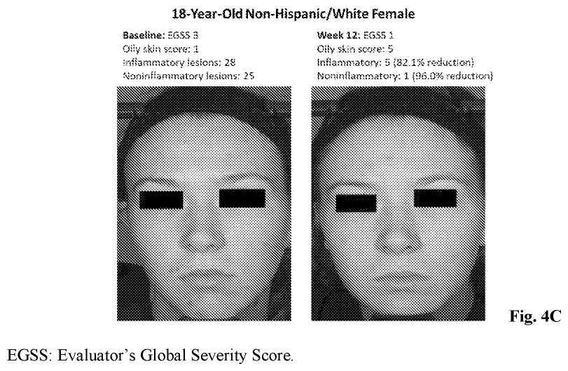

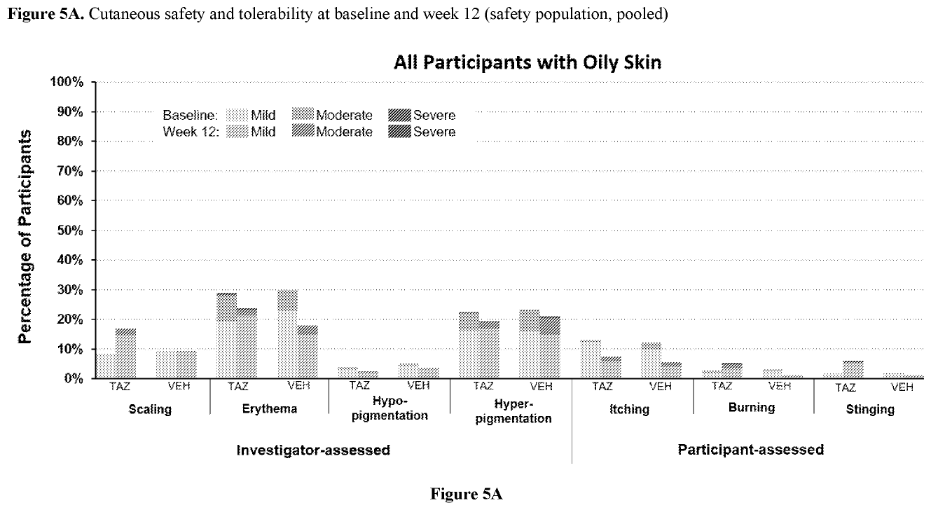

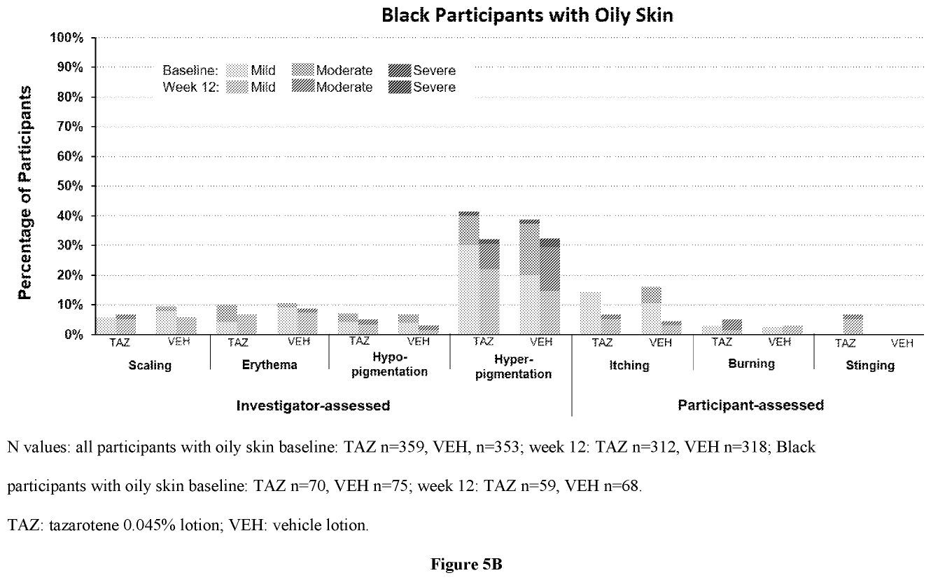

Skin oiliness and pore size may differ by race, with, as noted above, some studies demonstrating larger amounts of sebum secretion or larger pore sizes in Black patients and others, as noted above, finding no difference. Sebum composition and production can also vary by age and gender, and in patients with or without acne. The methods of the present invention can be used to treat subjects of all races, ages, genders, and skin types, e.g., white skin, light skin, dark skin, black skin, etc. It has surprisingly been found that the methods of the present invention are particularly effective for treating dark skin and black skin of subjects having both acne and oily skin. Such dark skin or black skin can be treated not only to improve acne, but also to improve or reduce skin oiliness. It has been found that by the end of treatment, Black subjects with oily skin saw improvements in skin oiliness, with oily skin assessments going from “oily” to “moderately” or “low/not” oily skin. As such, in one embodiment of the disclosed methods, the subject is a Black subject or a subject with darker skin, such as Hispanic, non-white subjects. The methods of the present invention improve acne and skin oiliness, while also providing improvements in hyperpigmentation (see, e.g., FIG. 4).

In another aspect, the present invention provides a method for treating truncal acne in a subject in need thereof. The method comprises topically applying to an affected area of the trunk of a subject suffering from acne any one of the compositions of the present invention, as disclosed herein, one or more (such as 1, 2, 3, or more) times per day for a period of time sufficient to treat such acne vulgaris. For example, such a period of time may be 1 to 30 days or longer as needed. For example, such a period of time may be one week, two weeks, four weeks, eight weeks, twelve weeks, or longer as needed. For example, a composition of the present invention is applied topically to affected areas of the body once per day for 7-14 days. Alternatively, it may be applied two or three times per day for 7-14 days. Alternatively, it may be applied once per day for one week to six months. For example, it may be applied once per day for two weeks, four weeks, eight weeks, or twelve weeks. In one embodiment, the treatment may be stopped for 1-7 days (e.g., 2, 3, 4, 5, 6, or 7 days) after an extended treatment period before it is resumed for another extended treatment period. Such an extended period may be 7 days, 7-14 days, 7-21 days, 7-30 days, or longer before more treatment is needed or desired. In one embodiment, the composition is a lotion composition as described herein, which is highly spreadable and, in turn, allows for more efficient delivery of tazarotene into the dermal layers.

In yet another aspect, the present invention provides a method of treating truncal acne with any of the pharmaceutical compositions of this invention, wherein the composition is applied once daily for more than 2 weeks, such as 4 weeks, for 6 weeks, for 8 weeks or for 12 weeks, without any serious adverse events, and/or with an improved adverse event profile compared to 0.1% tazarotene cream.

In yet another aspect, the present invention provides a method of treating truncal acne topically with any of the pharmaceutical compositions of this invention, wherein the composition is applied once daily for 12 weeks, and has better clinical efficacy and a lower adverse event profile as compared to commercial compositions containing 0.1% tazarotene (Tazorac® Cream).

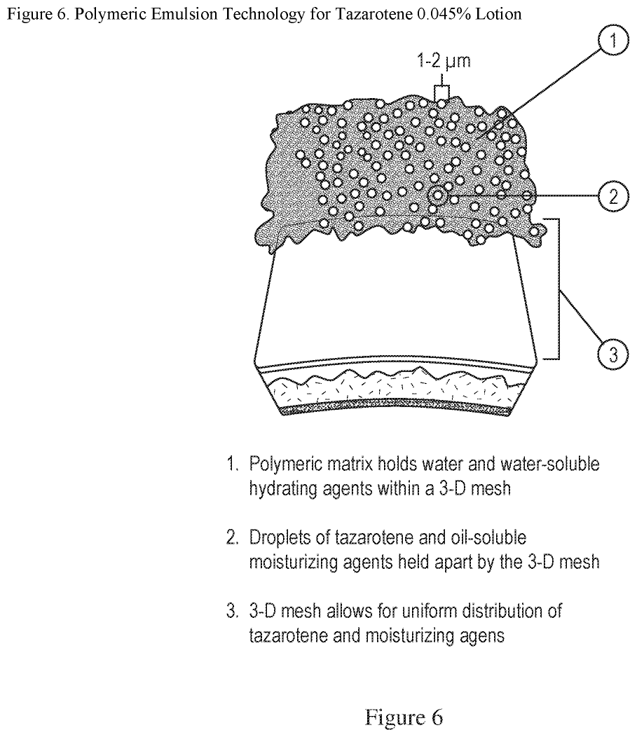

One of the advantages of the compositions used in all of the methods disclosed herein is that they employ the polymeric emulsion technology shown in FIG. 6. As shown therein, using the polymeric emulsion technology allows the following: (i) the polymeric matrix holds water and water-soluble hydrating agents within a 3-D matrix; (ii) the droplets of tazarotene and the oil-soluble moisturizing agents are held apart by the 3-D mesh; and (iii) the 3-D mesh allows for uniform distribution of tazarotene and moisturizing agents. Thus, the compositions of the present invention provide ease of spreadability, which is particularly important when treating truncal acne, which often involves treating larger areas where acne is present, rapid cutaneous penetration/effective drug delivery, and lack of residue on the skin, which is beneficial when treating either facial acne or truncal acne.

While the present disclosure shows and describes a number of exemplary embodiments, it will be manifest to those skilled in the art that various further modifications may be made without departing from the spirit and scope of the underlying inventive concept and that the same is not limited to particular compositions, processes, methods, or structures herein shown and described.

### EXAMPLES

### Example 1: Improvement in Acne and Skin Oiliness

### 1. Methods

a. Study Design and Participants

These analyses include data pooled from two previously described identically designed, phase 3, multicenter, double-blind, randomized, vehicle-controlled, parallel-group studies (NCT03168334 and NCT03168321) [Tanghetti EA I; and Tanghetti EA II)]. Briefly, eligible patients were aged ≥9 years with moderate-to-severe facial acne (a score of 3 or 4 on the Evaluator's Global Severity Score [EGSS]; Table 3, infra) and inflammatory and noninflammatory lesion counts between 20-50 and 25-100, respectively, with ≤2 facial nodules. Participants were equally randomized to tazarotene 0.045% lotion or vehicle lotion applied to the face once daily for 12 weeks.

TABLE 3
Evaluator's Global Severity Score

| Grade | Value | Severity |
|---|---|---|
| Clear | 0 | Normal, clear skin with no evidence of acne |
| Almost | 1 | Rare noninflammatory lesions present, with rare |
| clear | | noninflamed papules (papules must be resolving/may |
| | | be hyperpigmented, not pink-red) |
| Mild | 2 | Some noninflammatory lesions present, few |
| | | inflammatory lesions (papules/pustules only; no |
| | | nodulocystic lesions) |
| Moderate | 3 | Noninflammatory lesions predominate, multiple |
| | | inflammatory lesions; several/many comedones and |
| | | papules/pustules, and 0-1 nodulocystic lesions |
| Severe | 4 | Inflammatory lesions more apparent, many comedones |
| | | and papules/pustules, there may be 0-2 nodulocystic |
| | | lesions |

All studies were approved by relevant independent ethics committees or institutional review boards at each study site and were conducted in accordance with the International Conference on Harmonization, the Declaration of Helsinki, Good Clinical Practice Guidelines, and local regulations. All participants or their legal guardians provided written informed consent; participants under the legal age of consent provided assent.

b. Study Assessments

For each study, efficacy evaluations included investigator-assessed inflammatory lesions, noninflammatory lesions, and treatment success (defined as the proportion of participants achieving ≥2-grade reduction from baseline in EGSS and a score of 0 [clear] or 1[almost clear]). Self-reported changes in skin oiliness were also assessed using the Acne-Specific Quality of Life (Acne-QoL) questionnaire item 19, which is scored from 0 (extremely oily) to 6 (not at all oily; FIG. 1) [Martin AR, et al., Clin Exp Dermatol. 2001; 26(5): 380-385]. Safety evaluations comprised investigator-assessed cutaneous safety (scaling, erythema, hypopigmentation, hyperpigmentation) and participant-assessed tolerability (itching, burning, stinging) using a 4-point scale (0=none, 1=mild, 2=moderate, 3=severe). Adverse events (AEs) and serious adverse events (SAEs) were also monitored throughout the studies.

c. Statistical and Subgroup Analyses

For this post hoc analysis, participants were categorized by self-reported skin oiliness at baseline on the Acne-QoL item 19; only participants scoring 0-2 (oily skin; FIG. 1) at baseline were included. In addition, a subset of participants with oily skin who self-reported race as Black were analyzed separately. The intent-to-treat (ITT) population was defined as all participants who were randomized and received study drug. The safety population included all randomized participants who used study medication or vehicle at least once with a minimum of one post-baseline evaluation.

Least-squares (LS) mean percent changes from baseline in inflammatory and noninflammatory lesion counts by visit, treatment success at week 12, and changes in self-reported skin oiliness from baseline to week 12 were analyzed for all oily skin participants. The subgroup of Black participants with oily skin was also analyzed for changes in skin oiliness and cutaneous safety and tolerability. As significant skewness was found for lesion count changes, a nonparametric method was used to rank transform data prior to performing an analysis of covariance (ANCOVA), with a factor of treatment and the respective baseline lesion count as a covariate. Logistic regressions using Firth's Penalized Likelihood were performed to analyze treatment success, with a factor of treatment group. Statistical significance was defined as P≤0.05, determined using 2-tailed tests of the null hypothesis. Efficacy values were adjusted for multiple imputations. Missing data for lesion counts and treatment success were estimated using the Markov Chain Monte Carlo method. All statistical analyses were performed in SAS® version 9.3 or later. Dosing compliance (defined as participants missing ≤5 consecutive days of dosing and applying 80-120% of expected applications), changes in skin oiliness scores, and assessments of cutaneous safety and tolerability were summarized using descriptive statistics. AEs were recorded and classified using the Medical Dictionary for Regulatory Activities terminology. Imputations were not made for missing safety data.

### 2. Results

a. Participant Disposition and Demographics

Of 1614 participants in the intent-to-treat (ITT) population of the two pooled phase 3 studies, 1610 had data at baseline and week 12 on Acne-QoL question 19. Of these, 736 (45.7%) had oily skin at baseline and were included in these analyses. They had a mean age of 21.6 years, and the majority were female and White (Table 4, infra). Approximately 90% had an EGSS score of 3 (moderate) at baseline. Of 261 participants in the ITT population who identified as Black, 150 (57.4%) had oily skin. They were slightly older than the overall oily skin group (mean age: 25.2 years) and a greater percentage were female (83% versus 73%). The majority (94.7%) had moderate acne at baseline. Over 90% of all participants with oily skin and Black participants with oily skin were compliant in both treatment groups.

TABLE 4
Demographic and baseline characteristics of all participants
with oily skin (ITT population, pooled)

| | |
|---|---|
| Tazarotene | |
| 0.045% Lotion | Vehicle Lotion |
| (n = 369) | (n = 367) |

| | | | | |
|---|---|---|---|---|
| Age, mean (SD), y | 22.1 | (7.4) | 21.2 | (6.5) |
| Female, n (%) | 271 | (73.4) | 269 | (73.3) |
| Not Hispanic or Latino, n (%) | 282 | (76.4) | 267 | (72.8) |

Race, n (%)

| | | | | |
|---|---|---|---|---|
| White | 253 | (68.6) | 256 | (69.8) |
| Black | 72 | (19.5) | 78 | (21.3) |
| Asian | 23 | (6.2) | 17 | (4.6) |
| Other [symbol] | 21 | (5.7) | 16 | (4.4) |

Lesion counts, mean (SD)

| | | | | |
|---|---|---|---|---|
| Inflammatory | 28.2 | (7.2) | 27.8 | (6.9) |
| Noninflammatory | 41.5 | (16.6) | 41.6 | (16.6) |

Evaluator's Global Severity Score, n (%)

| | | | | |
|---|---|---|---|---|
| Moderate (score = 3) | 328 | (88.9) | 332 | (90.5) |
| Severe (score = 4) | 41 | (11.1) | 35 | (9.5) |

[symbol]

Includes American Indian or Alaska Native, Native Hawaiian or Other Pacific Islander, and other/multiple races.
ITT: intent to treat;
SD: standard deviation [symbol]
indicates data missing or illegible when filed

b. Efficacy

In all participants with oily skin, tazarotene 0.045% lotion provided over 55% reductions in lesion counts from baseline to week 12. These reductions were significantly greater than those observed with vehicle lotion in inflammatory (LS means: −57.0% vs −48.4%; P<0.001) and noninflammatory lesion counts (−55.9% vs −42.1%; P<0.001; FIG. 2). Treatment success rates at week 12 were significantly higher for all tazarotene-treated oily skin participants compared with vehicle, with almost a third of tazarotene-treated participants achieving treatment success (29.8% vs 19.2%; P<0.01). These acne improvements are similar to those seen in the overall phase 3 pooled population for lesion reductions (tazarotene versus vehicle; inflammatory: −57.9% vs −47.8%; noninflammatory: −56.0% vs −42.0%; P<0.001, both) and treatment success rates at week 12 (30.4% vs 17.9%; P<0.001) [Tanghetti EA I].

Over 70% of participants in the overall oily skin population reported an improvement in skin oiliness to “moderately” or “low/not” oily with both tazarotene 0.045% lotion and vehicle; a greater percentage of participants reported an improvement to “low/not” oily skin with tazarotene than vehicle (FIG. 3). Compared with the overall oily skin population, a higher percentage of Black participants with oily skin saw improvements in skin oiliness. Over three quarters of tazarotene-treated Black participants reported an improvement in skin oiliness to “moderately” or “low/not” oily skin, compared with about two thirds of vehicle-treated participants (FIG. 3). Images depicting acne improvements with tazarotene 0.045% lotion are shown in FIG. 4.

c. Safety

Treatment-emergent AE (TEAE) rates with tazarotene in all oily-skin participants (Table 5, infra) were similar to those observed in the overall tazarotene-treated population (n=779; any TEAE: 27.9% vs 26.8% overall; treatment-related TEAE: 11.7% vs 11.3% overall) [Tanghetti EA II]. Most TEAEs were mild in severity and the majority were considered unrelated to treatment. A total of 1.7% of tazarotene-treated participants with oily skin discontinued the study or study drug compared with 2.8% of tazarotene-treated participants overall [Tanghetti EA II].

TABLE 5
Treatment-emergent adverse events through week 12 in all
participants with oily skin (safety population, pooled)

| | Tazarotene | | | |
|---|---|---|---|---|
| | 0.045% Lotion | | Vehicle Lotion | |
| n (%) | (n = 359) | | (n = 353) | |
| Any TEAE | 100 | (27.9) | 64 | (18.1) |
| Any serious TEAE [symbol] | 2 | (0.6) | 2 | (0.6) |
| Discontinuations due to TEAEs | 6 | (1.7) | 3 | (0.8) |

Severity of TEAEs reported

| | | | | |
|---|---|---|---|---|
| Mild | 72 | (20.1) | 36 | (10.2) |
| Moderate | 23 | (6.4) | 25 | (1.7) |
| Severe | 5 | (1.4) | 3 | (0.8) |

Relationship to study drug

| | | | | |
|---|---|---|---|---|
| Related | 42 | (11.7) | 7 | (2.0) |
| Unrelated | 58 | (16.2) | 57 | (16.1) |

[symbol]

None of the participants had serious AEs that ware considered to be treatment related.
TEAE, treatment-emergent adverse event. [symbol]
indicates data missing or illegible when filed

For the cutaneous safety and tolerability assessments, most (≥71%) tazarotene-treated participants with oily skin reported ratings of 0 (none) at baseline and week 12; of those participants who had any signs/symptoms at baseline or week 12, fewer than 1% reported ratings of 3 (severe; FIG. 5). As expected, transient increases in mild-to-moderate itching, burning, stinging, scaling, and erythema were observed at weeks 2-8 (data not shown) and mirrored results observed in the overall population [Tanghetti EA II]. Only erythema and hyperpigmentation were reported in >20% of participants in either treatment group at baseline (28.9% and 22.5%, respectively); by week 12, fewer tazarotene-treated participants reported any erythema (23.7%) or hyperpigmentation (19.5%; FIG. 5). Similar trends were observed in the tazarotene-treated Black participants with oily skin (n=145), though there were greater improvements in hyperpigmentation by week 12 than the overall oily skin population (hyperpigmentation at baseline and week 12:41.4% and 32.2% Black subgroup versus 22.6% and 19.6% overall oily skin). Further, Black participants with oily skin had fewer reports of erythema and scaling at baseline or week 12 than the overall population with oily skin (FIG. 5).

### 3. Discussion

Excessive sebum production is one of several factors involved in the pathophysiology of acne [Jappe U. Acta Derm Venereol. 2003; 83(4): 241-248; and Zaenglein A L, et al., J Am Acad Dermatol. 2016; 74(5): 945-973.e933]. These post hoc analyses of data pooled from two phase 3 trials showed that tazarotene 0.045% polymeric emulsion lotion, applied once daily for 12 weeks, improved acne symptoms and perceived skin oiliness and was well tolerated in participants with oily skin and moderate-to-severe acne, including Black participants.

In the overall oily skin population, tazarotene 0.045% lotion provided over 55% reductions in inflammatory and noninflammatory lesion counts at week 12. Earlier improvements than with vehicle were also demonstrated, with significantly greater reductions from baseline noted at weeks 4 and 8 in noninflammatory and inflammatory lesion counts, respectively. In addition, almost one third of tazarotene-treated participants achieved treatment success at week 12 versus less than one quarter of those treated with vehicle. These results are similar to those observed in the previously published overall pooled phase 3 studies [Tanghetti EA I], and demonstrate that tazarotene 0.045% lotion is also efficacious in patients with acne and oily skin.

As facial acne and oily skin can both negatively affect quality of life [Arbuckle R, et al., Health Qual Life Outcomes. 2008; 6:80; Wu Y, et al., Int J Cosmet Sci. 2013; 35(5): 442-447; Skroza N, et al. J Clin Aesthet Dermatol. 2018; 11(1): 21-25; and Kircik L H, et al. J Drugs Dermatol. 2020; 19(11): 1086-1092], an efficacious acne treatment that can also reduce skin oiliness is beneficial and advantageous to those with both acne and oily skin. Some acne treatments, such as topical retinoids [Zouboulis C C, et al. Rev Endocr Metab Disord. 2016; 17(3): 319-334 (hereinafter, “Zouboulis C C, et al.”] or topical clascoterone 1% cream (androgen receptor inhibitor) [Hebert A, et al. JAMA Dermatol. 2020; 156(6): 621-630], are postulated to have a sebum suppressing effect, but clinical data supporting this have not been published. In the tazarotene 0.045% phase 3 studies, almost half (45.7%) of participants with moderate-to-severe acne reported having oily skin at baseline, while only 16.0% reported having low or not oily skin. Importantly, by week 12, almost three quarters of all participants with oily skin improved to “moderately” or “low/not” oily skin with tazarotene 0.045% lotion or vehicle lotion.

These are the first published results on the impact of a topical retinoid on oily skin in acne patients. Interpretation of these clinical data can be challenging, however, given limitations in study design and analysis. These phase 3 studies were not powered for oily skin analyses, though this limitation exists for all post hoc analyses from clinical trials. Additionally, skin oiliness was measured via participant self-report as an item from the Acne-QoL questionnaire, which is a tool validated to measure overall patient quality of life and not oily skin specifically. Finally, participants may have changed their skin care routine while enrolled in the studies (ie, cleansed/moisturized more or less often).

Nevertheless, without intending to be bound by any theory, improvements in oiliness with tazarotene lotion observed in these analyses is believed to be due in part to inhibition of sebocyte differentiation or lipid synthesis [Zouboulis C C, et al.] and/or more likely the unique, non-greasy excipients/emulsifiers contained in the polymeric emulsion lotion vehicle. This emulsion technology of the compositions used in the methods of the present invention provides fast, uniform, and complete release of the humectants, oil droplets, and other excipients contained in the vehicle onto the skin [Tanghetti EA III]. Indeed, improvements in skin oiliness were similar with both tazarotene and vehicle lotion in the overall oily skin population, with 71% of participants in both treatment groups experiencing improvements. This suggests that the emulsification process, which releases the excipients and mixes with the surface lipids, results in a less oily and greasy feel and appearance of the skin even with the enhanced hydration provided by the added moisturizers. The exact nature, chemistry, and rheology of this process is still under investigation. Reductions in oiliness with tazarotene 0.045% lotion are particularly of interest given that topical retinoid use is associated with irritation and dryness [Leyden J, et al., Dermatol Ther (Heidelb). 2017; 7(3): 293-304].

Sebum production varies greatly between individuals, but factors that may affect skin oiliness include sex, race, and climate [Endly DC, et al., J Clin Aesthet Dermatol. 2017; 10(8): 49-55]. Although males typically produce more sebum than females [Luebberding S, et al., Int J Cosmet Sci. 2013; 35(5): 477-483; and Rahrovan S, et al., Int J Womens Dermatol. 2018; 4(3): 122-130], it is noteworthy that in the current analyses there were more females in the oily skin group than in the overall population (73% vs 66%, respectively) [Tanghetti EA I]. It is possible that women find oily skin to be more troublesome than men and were therefore more likely to report greater skin oiliness at baseline. Studies have shown that women with acne tend to report worse quality of life than men with acne [Skroza N, et al., J Clin Aesthet Dermatol. 2018; 11(1): 21-25; and Kircik L H, et al., J Drugs Dermatol. 2020; 19(11): 1086-1092 (hereinafter, “Kircik L H”], though it is unclear how much of this is attributable to skin oiliness. A separate post hoc analysis of data pooled from these phase 3 studies of tazarotene 0.045% lotion showed that females had worse acne-related quality of life scores at baseline and greater improvements after 12 weeks of tazarotene treatment than males [Kircik L H]. In the present studies, tazarotene 0.045% lotion improved perceived skin oiliness over 12 weeks; quality of life, however, was not analyzed for this subgroup of participants with oily skin. There is little published data on the impact of topical retinoids on oily skin and quality of life, and thus, this topic may warrant further research.

In the current analyses, tazarotene 0.045% lotion demonstrated good safety and tolerability in participants with oily skin. Rates of TEAEs and treatment-related TEAEs were similar to those observed in the overall tazarotene-treated population [Tanghetti EA I], and fewer than 2% of tazarotene-treated participants with oily skin discontinued the study or study drug. Over 70% of participants with oily skin treated with tazarotene lotion reported scores of none (0) on all cutaneous safety and tolerability assessments at baseline and week 12, and any increases in severity at weeks 2-8 were transient.

Although acne pathophysiology is believed to be the same across ethnic/racial groups, differences in skin structure and acne-related sequelae such as post-inflammatory hyperpigmentation and scarring may require a more individualized treatment approach, including assessment of racial characteristics and skin color [Alexis A F, et al., J Drugs Dermatol. 2021; 20(7): 716-725]. When Black participants with oily skin were analyzed separately in the current analyses, over three-quarters treated with tazarotene reported an improvement in skin oiliness to “moderately” or “low/not” oily skin. While the overall oily skin population showed similar improvements with tazarotene and vehicle, a numerically greater percentage of Black participants experienced improvements with tazarotene versus vehicle (81.4% versus 67.6%). In terms of cutaneous safety and tolerability, tazarotene-treated Black participants with oily skin showed similar trends to the overall oily skin population; however, improvements in hyperpigmentation were greater in Black participants by week 12 and there were fewer reports of erythema and scaling at baseline or week 12. These results mirror those from the pooled phase 3 studies which demonstrated that tazarotene 0.045% lotion is efficacious and safe in Black participants with moderate-to-severe acne, with reductions in inflammation-related sequalae such as erythema and hyperpigmentation [Bhatia N, et al., J Drugs Dermatol. 2020; 19(7): 727-734].

### 4. Conclusions

In two pooled phase 3 studies, nearly three fourths of participants with oily skin treated with tazarotene 0.045% polymeric emulsion lotion—including Black participants—had subjective reductions in skin oiliness by week 12, with over a third reporting low or not oily skin. Tazarotene lotion also showed efficacy and safety in the treatment of moderate-to severe acne in participants with oily skin, with outcomes similar to the overall study population. Once-daily treatment with tazarotene 0.045% lotion may help to improve patient-perceived skin oiliness in those with moderate-to-severe acne.

### Example 2: Treatment of Truncal Acne: Efficacy, Safety, and Spreadability

### 1. Introduction

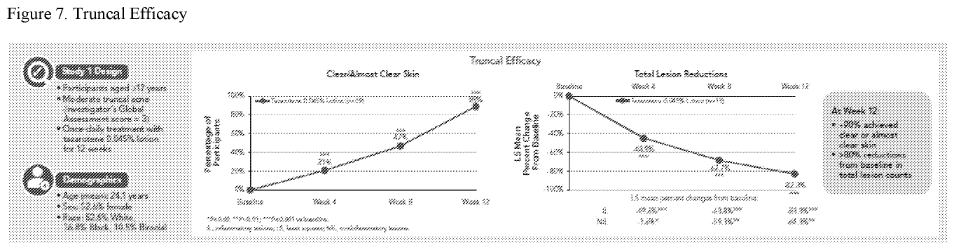

Truncal acne—occurring on the chest and/or back—is common among patients with facial acne. Topical treatment of truncal acne is complicated by the involvement of a large body surface area, necessitating formulations that are highly spreadable, non-irritating, and provide rapid drug delivery. Tazarotene 0.045% lotion (TAZ) was developed using polymeric emulsion technology to provide uniform and rapid distribution of tazarotene and moisturizing/hydrating excipients in a highly spreadable formulation (FIG. 6). Here, the efficacy, safety, and tolerability of TAZ in the treatment of truncal acne as well as its irritation potential and spreadability on the trunk are summarized in FIGS. 7-10.

### 2. Methods

In a 12-week phase 4, open-label study, participants (≥12 years; N=19) with moderate truncal acne (Investigator's Global Assessment [IGA] score=3) were treated with once-daily TAZ. Outcomes included IGA score, lesion counts, cutaneous tolerability, and adverse events (AEs). Irritation potential of TAZ was evaluated in phase 1 repeat insult (RIPT; N=210) and cumulative irritation (CIPT; N=42) patch tests, in which dermal responses to repeated placement of patches loaded with TAZ, vehicle lotion, or controls were assessed on the upper back. In a double-blind, split-body study (N=30), spreadability was compared for TAZ and trifarotene 0.005% cream applied to participants' backs.

### 3. Results

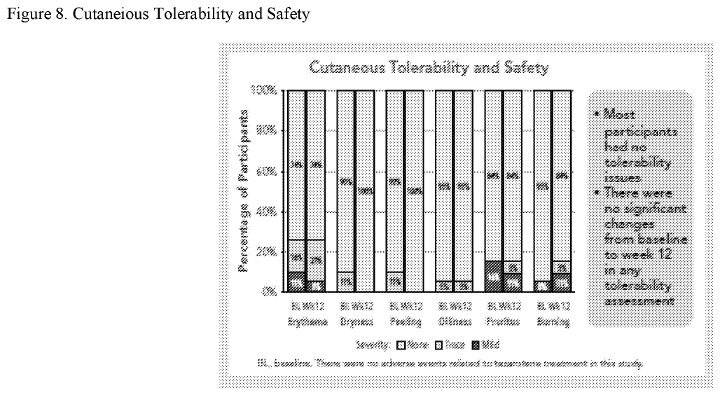

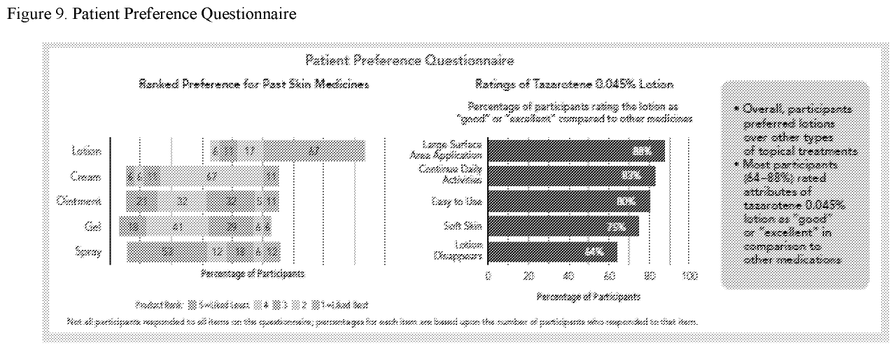

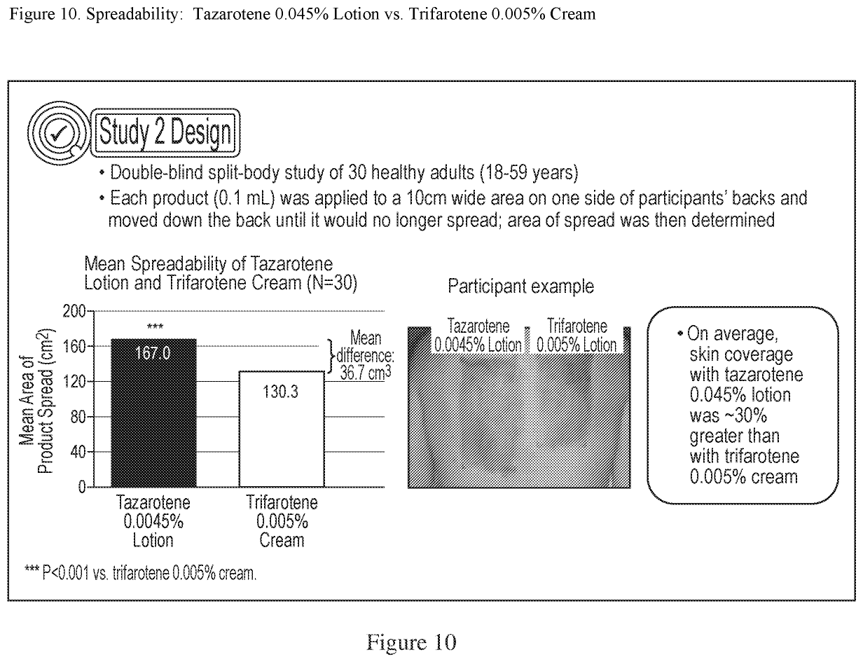

After 12 weeks of treatment with TAZ, 89% of participants (17/19) achieved clear/almost clear truncal skin (IGA score of 0 or 1; P<0.001 vs baseline) (FIG. 7A). Large least-squares mean percent reductions from baseline in inflammatory, noninflammatory, and total lesion counts were also observed (83%, 64%, and 82%, respectively; P<0.01, all) (FIG. 7B). Significant improvements from baseline in IGA score and lesion counts were observed as early as week 4. There were no AEs related to TAZ treatment. At baseline and week 12, most participants (≥74%) had no tolerability issues, and there were no significant changes from baseline to week 12 in any tolerability assessment (erythema, dryness, peeling, oiliness, pruritus, and burning) (FIG. 8). In the RIPT and CIPT studies, TAZ demonstrated a low potential for irritancy/contact dermatitis and did not induce sensitization under exaggerated dosing conditions. Moreover, in the Patient Preference Questionnaire, participants preferred lotions to other types of topical treatments, such as creams, ointments, gels, and sprays. In addition, most participants (64% to 88%) rated attributes of TAZ, i.e., tazarotene 0.045% lotion, as “good” or “excellent” in comparison to other medications (FIG. 9). In the split-body study, TAZ covered on average ˜30% more skin than the same amount of trifarotene cream (FIG. 10).

### 4. Conclusions

Tazarotene 0.045% lotion significantly reduced truncal acne lesions after 12 weeks of once-daily use, and demonstrated low irritation potential in patients with truncal acne and in patch tests. This easy-to-apply lotion—utilizing polymeric emulsion technology to improve drug delivery and limit irritation—had greater skin coverage compared with trifarotene cream. Overall, tazarotene 0.045% lotion is an effective and well tolerated option for the treatment of truncal acne, with sensory and aesthetic properties preferred by patients.

### Example 3: Acne Clinical Trial #1

A first clinical study in acne patients was conducted to compare the efficacy of a composition of the present invention containing tazarotene (the “Composition A” lotion of Table 2).

A first placebo (“Lotion Placebo”) corresponded to and had a similar viscosity as Composition A but lacked tazarotene. A second placebo (“Cream Placebo”) corresponded to the Lotion Placebo but employed carbomer homopolymer type C in place of carbomer homopolymer type A to yield a higher viscosity emulsion with a viscosity similar to 0.1% tazarotene cream employed in the study. This allows for better blinding of the test formulations and control of the clinical study. In the following results, data for Lotion Placebo and Cream Placebo may be combined and reported as “Combined Placebo”.

Additionally, commercially available 0.1% tazarotene cream (Tazorac® 0.1% cream) was employed in this clinical study for comparison purposes.

A double blind, multi-site, randomized clinical study was conducted on patients suffering from acne vulgaris, whereby neither the acne patient nor the investigator knew the identity of the test composition assigned. 210 patients were randomized to receive either Composition A lotion, Tazorac® 0.1% cream, Placebo Lotion and Placebo Cream at a 2:2:1:1 randomization:
69 subjects “Composition A lotion”
72 subjects “Tazorac® 0.1% cream”
34 subjects “Lotion Placebo”
35 subjects “Cream Placebo”.

Primary inclusion criteria were moderate to severe acne, inflammatory lesion count of at least 20 but no more than 40, and non-inflammatory lesion count of at least 20 but no more than 100. The blindly labeled lotions were applied to the affected area once daily for twelve weeks, with assessments at 2 weeks, 4 weeks, 8 weeks and 12 weeks.

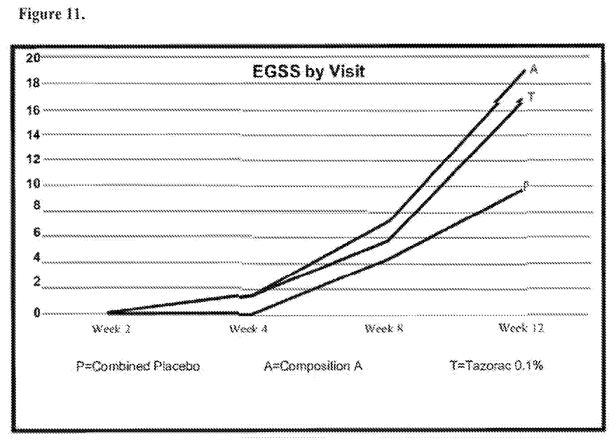

The investigator monitored the efficacy at each study visit by assessing the treatable area, determining the Evaluator's Global Severity Score (EGSS), and determining the grade of improvement (see, FIG. 11). EGSS included the following grades:

$$Clear = 0 ; Almost Clear = 1 ; Mild = 2 ; Moderate = 3 ; Severe = 4.$$

Clinical Efficacy was determined primarily based on the percentage of subjects who were treatment successes at 12 weeks. To be judged as a treatment success, as reported in Tables 7, 9, and 10, subjects had to show at least two-grade improvement from the baseline and EGSS score equating to “clear” or “almost clear”. Also, the numbers of inflammatory and non-inflammatory lesions were assessed.

Table 6 reports Baseline Characteristics-Inflammatory Lesions and Non-Inflammatory Lesions:

| | Combined | | Tazorac ® |
|---|---|---|---|
| | Placebo | Composition A | 0.1% Cream |
| | N = 69 | N = 69 | N = 72 |
| Inflammatory Lesions | | | |
| Mean (SD) | 27.2 (5.49) | 28.3 (6.0) | 27.3 (5.95) |
| Median | 26.0 | 27.0 | 26.0 |
| Non-inflammatory Lesions | | | |
| Mean (SD) | 36.6 (13.17) | 37.6 (14.70) | 36.6 (13.31) |
| Median | 34.0 | 34.0 | 34.0 |

Table 7 reports treatment % success on an intention-to-treat (ITT) basis.

EGSS (Clear or Almost Clear) by Visit (ITT)

| | Combined | | Tazorac ® |
|---|---|---|---|
| | Placebo | Composition A | 0.1% Cream |
| | N = 69 | N = 69 | N = 72 |
| Week 2 | 0% | 0% | 0% |
| Week 4 | 0% | 1.4% | 1.4% |
| Week 8 | 4.3% | 7.2% | 5.6% |
| Week 12 | 10.1% | 18.8% | 16.7% |

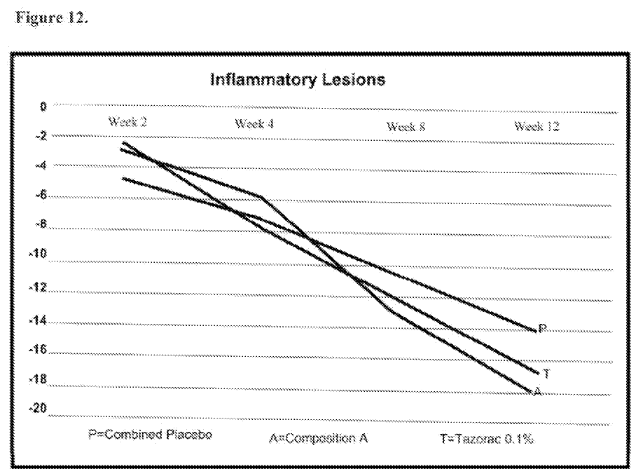

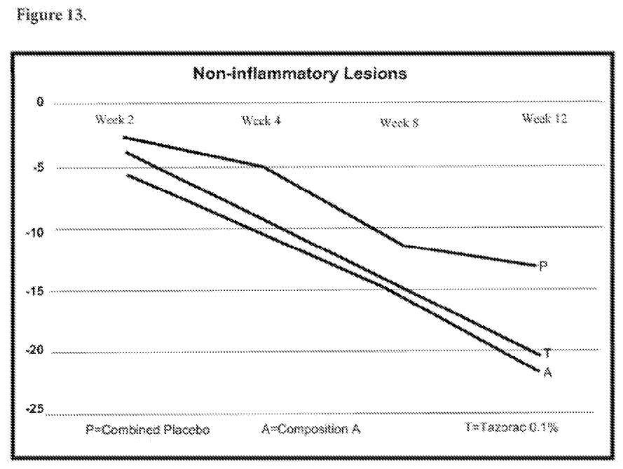

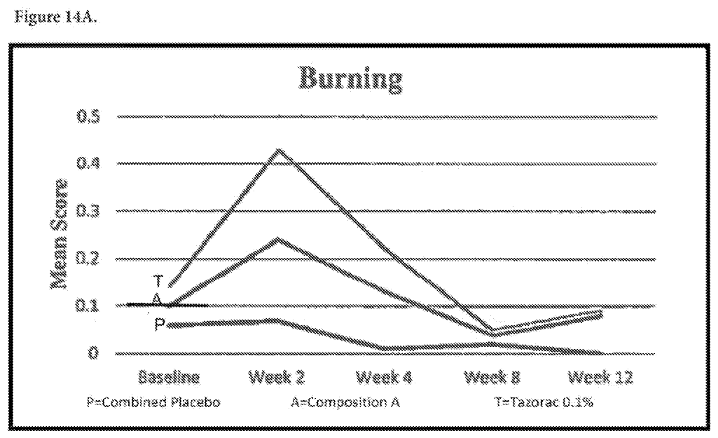

Table 8 reports absolute change in inflammatory lesions from baseline. Change in inflammatory and non-inflammatory lesions from baseline is reported in FIGS. 12 and 13.

| | Combined | Composition A | Tazorac ® 0.1% |
|---|---|---|---|
| | Placebo N = 69 | N = 69 | Cream N = 72 |
| Week 2 | −4.7 | −2.9 | −2.4 |
| Week 4 | −7.2 | −5.8 | −7.8 |
| Week 8 | −10.9 | −13.2 | −12.0 |
| Week 12 | −14.0 | −18.1 | −16.8 |

Table 9 reports treatment % success for primary efficacy on an intention-to-treat basis for EGSS at week 12. For EGSS, to be judged as a treatment success, subjects had to show at least two-grade improvement from the baseline and EGSS score equating to “clear” or “almost clear”. For inflammatory and non-inflammatory lesions, absolute change from baseline to week 12 in mean inflammatory and non-inflammatory counts is reported.

| | Combined | Composition A | Tazorac ® 0.1% |
|---|---|---|---|
| | Placebo N = 69 | N = 69 | Cream N = 72 |
| EGSS - Clear | 10.1% | 18.8% | 16.7% |
| or Almost Clear | | | |
| Inflammatory | | | |
| Lesions | | | |
| Absolute Change | −14.0 | −18.1 | −16.8 |
| % Change | −51.41% | −63.75% | −59.99% |
| Non-inflammatory | | | |
| Lesions | | | |
| Absolute Change | −13.1 | −21.6 | −20.3 |
| % Change | −35.18% | −56.86% | −54.09% |

Table 10 reports treatment % success on a per-protocol basis for EGSS. For lesions, absolute change from baseline to Week 12 in mean inflammatory and non-inflammatory counts is reported.

| | Combined | Composition A | Tazorac ® 0.1% |
|---|---|---|---|
| | Placebo N = 57 | N = 54 | Cream N = 56 |
| EGSS - Clear | 12.3% | 20.4% | 16.1% |
| or Almost Clear | | | |
| Inflammatory | −14.2 | −18.3 | −18.2 |
| Lesions | | | |
| Non-inflammatory | −11.8 | −20.8 | −21.9 |
| Lesions | | | |

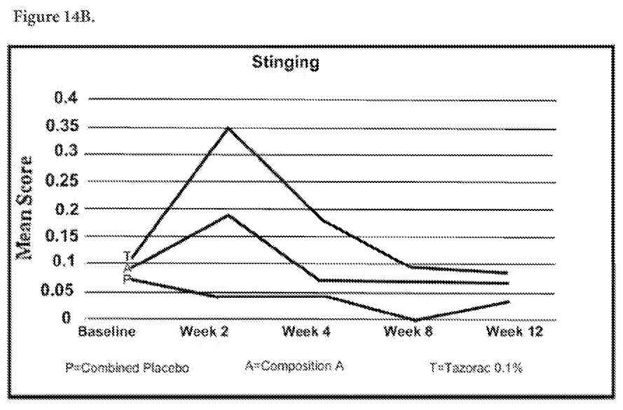

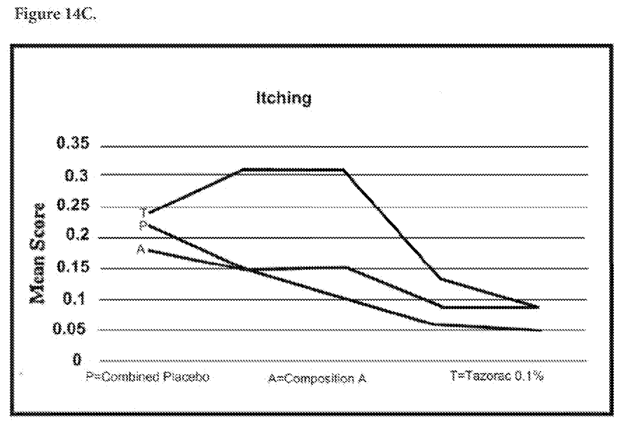

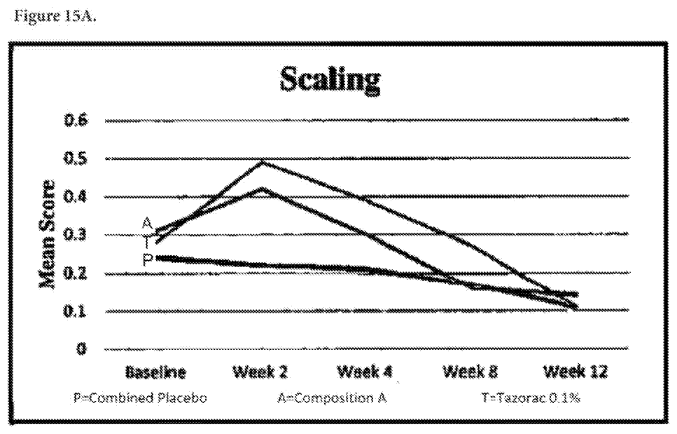

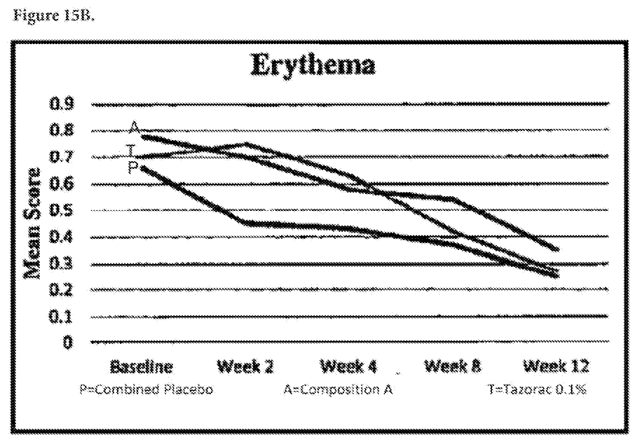

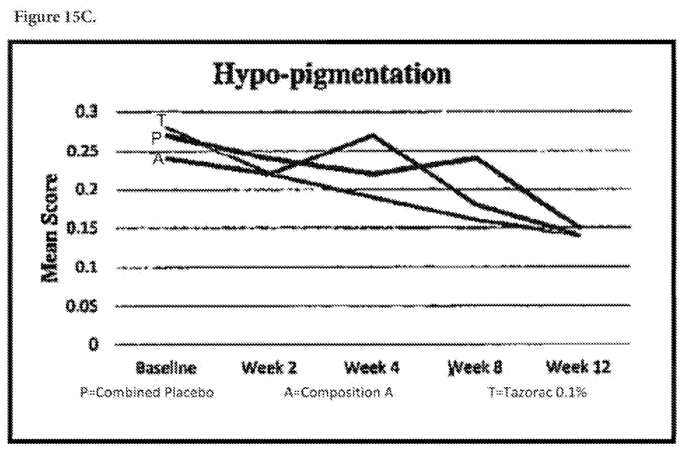

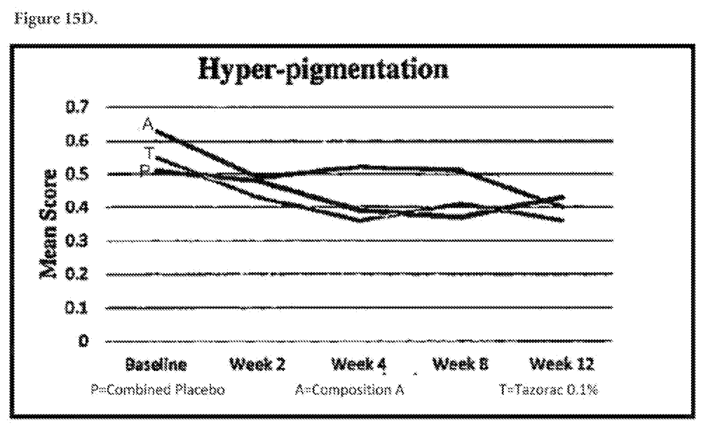

The compositions were tested for cutaneous safety and tolerability. Cutaneous tolerability was assessed by burning, stinging or itching reported by the subjects. This data is summarized in FIG. 14. Cutaneous safety was assessed by scaling, erythema, hypo-pigmentation and hyper-pigmentations as evaluated by the investigator. This data is summarized in FIG. 15.

Table 11 reports treatment emergent adverse event characteristics.

| | Combined | | Tazorac ® 0.1% |
|---|---|---|---|
| | Placebo | Composition A | Cream |
| % Subjects | 13.4% | 14.7% | 26.8% |
| Reporting Any | | | |
| Adverse Event | | | |
| % Subjects | 0% | 0% | 0% |
| Reporting Any | | | |
| Serious Adverse | | | |
| Event | | | |
| % Subjects | 0% | 0% | 1.4% |
| Discontinued | | | |
| Study Due to | | | |
| Adverse Event | | | |

Notably, this clinical study showed that Composition A, containing less than half of the tazarotene active ingredient, had a numerically better efficacy than the Tazorac® 0.1% Cream. Also, the adverse event profile was lower for Composition A than for Tazorac® 0.1% Cream.

### Example 4: Acne Clinical Trial #2 and 3

Two larger clinical studies (Studies 301 and 302) in acne patients were conducted to compare the efficacy of a composition of the present invention containing tazarotene (the “Composition A” Lotion of Table 2) to that of a placebo that was the vehicle of Composition A (i.e., without the active ingredient tazarotene).

These studies were phase-3, multi-center, randomized, double-blind, vehicle-controlled, two-arm, parallel group studies comparing the safety and efficacy of Composition A (“IDP-123 Lotion” or “Composition A Lotion”) and IDP-123 vehicle lotion (“Placebo Lotion”) in the treatment of acne vulgaris. Neither the acne patient nor the investigator knew the identity of the test composition assigned.

In the first study (Study 301), 813 patients were randomized to receive either Composition A Lotion or Placebo Lotion, as follows.
402 subjects receiving “Composition A Lotion,”
411 subjects receiving “Placebo Lotion.”

In the second study (Study 302), 801 patients were randomized to receive either Composition A Lotion or Placebo Lotion, as follows.
397 subjects receiving “Composition A Lotion,”
404 subjects receiving “Placebo Lotion.”

Primary inclusion criteria were moderate to severe acne, inflammatory lesion count of at least 20 but no more than 50, and non-inflammatory lesion count of at least 25 but no more than 100. The blindly labeled lotions were applied to the affected facial area once daily for twelve weeks, with assessments at 2 weeks, 4 weeks, 8 weeks and 12 weeks.

The investigator monitored the efficacy at each study visit by assessing the treatable area, determining the Evaluator's Global Severity Score (“EGSS”), and determining the grade of improvement. EGSS included the following grades:

$$Clear = 0 ; Almost Clear = 1 ; Mild = 2 ; Moderate = 3 ; Severe = 4.$$

Clinical Efficacy was determined primarily based on: (1) superiority in absolute change from baseline to week 12 in mean inflammatory counts; (2) superiority in absolute change from baseline to week 12 in mean non-inflammatory counts; and (3) percentage of subjects who achieve at least a two-grade reduction from baseline and are “Clear” or “Almost Clear” at week 12 in the EGSS.

Table 12 reports Subject Baseline Characteristics-Inflammatory Lesions and Non-inflammatory Lesion Counts. The first part of Table 12 reports treatment % success at week 12 on intention-to-treat:

| | Study 301 | | | | Study 302 | | | |
|---|---|---|---|---|---|---|---|---|
| | Composition | | Placebo | | Composition | | Placebo | |
| | A Lotion | | Lotion | | A Lotion | | Lotion | |
| | N = 402⁽¹⁾ | | N = 411⁽¹⁾ | | N = 397⁽¹⁾ | | N = 404⁽¹⁾ | |
| Inflammatory | | | | | | | | |
| Lesions | | | | | | | | |
| Mean (SD) | 28.5 | (7.04) | 28.1 | (7.04) | 28.0 | (7.32) | 27.9 | (7.10) |
| Median | 26.0 | | 26.0 | | 26.0 | | 26.0 | |
| Non-inflammatory | | | | | | | | |
| Lesions | | | | | | | | |
| Mean (SD) | 41.1 | (15.67) | 40.7 | (16.29) | 41.8 | (17.87) | 40.6 | (16.31) |

| | | | | |
|---|---|---|---|---|
| Median | 36.0 | 34.0 | 36.0 | 35.0 |

(ITT) basis.

The second part of Table 12 EGSS (Clear or Almost Clear) at Week 12 (ITT):

| | Study 301 | | Study 302 | |
|---|---|---|---|---|
| | Composition A | Placebo | Composition | Placebo |
| | Lotion | Lotion | A Lotion | Lotion |
| | N = 402 | N = 411 | N = 397 | N = 404 |
| Week 12 | 25.5% | 13.0% | 29.6% | 17.3 |
| | (p < 0.001) | | (p < 0.001) | |

Table 13 reports absolute changes in inflammatory and non-inflammatory lesions from baseline at Week 12.

| | Study 301 | | Study 302 | |
|---|---|---|---|---|
| | Composition A | Placebo | Composition A | Placebo |
| | Lotion | Lotion | Lotion | Lotion |
| | N = 402 | N = 411 | N = 397 | N = 404 |
| Inflammatory | −15.6 | −12.4 | −16.7 | −13.4 |
| Lesions | (p < 0.001) | | (p < 0.001) | |
| Non- | −21.0 | −16.4 | −24.6 | −16.6 |
| inflammatory | (p < 0.001) | | (p < 0.001) | |
| Lesions | | | | |

Table 14 reports assessment of treatment success on a per-protocol basis for EGSS. For lesions, absolute changes from baseline to week 12 in mean inflammatory and non-inflammatory counts are reported.

| | Study 301 | | Study 302 | |
|---|---|---|---|---|
| | Composition | Placebo | Composition | Placebo |
| | A Lotion | Lotion | A Lotion | Lotion |
| | N = 402 | N = 411 | N = 397 | N = 404 |
| EGSS - | 26.8% | 14.4% | 30.6% | 18.3% |
| Clear or Almost Clear | | | | |
| Inflammatory Lesions | −15.8 | −12.4 | −16.4 | −13.4 |
| Non-inflammatory | −21.3 | −16.3 | −24.1 | −15.4 |
| Lesions | | | | |

The compositions were tested for cutaneous safety and tolerability. Cutaneous tolerability was assessed by itching, burning, or stinging reported by the subjects. Cutaneous safety was assessed by scaling, erythema, hypo-pigmentation, and hyper-pigmentations, as evaluated by the investigator.

Table 15 shows the number of subjects reporting moderate or severe itching, burning, or stinging; or being observed to have moderate or severe scaling, erythema, hypopigmentation, or hyperpigmentation. In general, the reported numbers of these conditions are similar between those subjects using Composition A Lotion and Placebo Lotion.

Cutaneous Safety and Tolerability

| | Study 301 | | Study 302 | |
|---|---|---|---|---|
| | Composition A | Placebo | Composition A | Placebo |
| | Lotion | Lotion | Lotion | Lotion |
| | N = 387 | N = 392 | N = 392 | N = 399 |
| Scaling | | | | |
| Moderate | 4 | 2 | 6 | 1 |
| Severe | 0 | 0 | 1 | 0 |
| Erythema | | | | |
| Moderate | 7 | 8 | 13 | 6 |
| Severe | 0 | 0 | 1 | 0 |
| Hypopigmentation | | | | |
| Moderate | 1 | 1 | 0 | 0 |
| Severe | 0 | 0 | 0 | 0 |
| Hyperpigmentation | | | | |
| Moderate | 7 | 9 | 8 | 17 |
| Severe | 1 | 2 | 0 | 1 |
| Itching | | | | |
| Moderate | 4 | 2 | 4 | 4 |
| Severe | 0 | 0 | 0 | 0 |
| Burning | | | | |
| Moderate | 2 | 0 | 5 | 0 |
| Severe | 0 | 0 | 2 | 0 |
| Stinging | | | | |
| Moderate | 1 | 1 | 3 | 0 |
| Severe | 0 | 0 | 1 | 0 |

Table 16 reports on treatment emergent adverse event characteristics.

Treatment Emergent Adverse Event Characteristics

| | Study 301 | | Study 302 | |
|---|---|---|---|---|
| | Composition | Placebo | Composition | Placebo |
| | A Lotion | Lotion | A Lotion | Lotion |
| | N = 392⁽¹⁾ | N = 399⁽¹⁾ | N = 387⁽¹⁾ | N = 392⁽¹⁾ |
| % Subjects | 25.5% | 19.5% | 28.2% | 18.6% |
| Reporting Any | | | | |
| Adverse Event | | | | |
| % Subjects | 0.8% | 0.8% | 0.3% | 0.3% |
| Reporting Any | | | | |
| Serious Adverse | | | | |
| Event | | | | |
| % Subjects | 3.1% | 0.5% | 2.6% | 0.5% |
| Discontinued | | | | |
| Study Due to | | | | |
| Adverse Event | | | | |

Note:
⁽¹⁾safety population, which comprises all randomized subjects who were presumed to have used the study drug at least once and who provided at least one post-baseline safety evaluation.

Notably, these clinical studies showed that the percentages of subjects reporting any serious adverse event for Composition A are no higher than those for the Placebo Lotion, indicating the excellent safety of Composition A.

The treatment success, as measured by investigator's global assessment of Composition A Lotion (denoted as “EGSS” above) and some commercial products containing a retinoid (as gleaned from their package inserts) is shown in Table 17.

Comparison of Treatment Success of Various
Products Containing a Retinoid

| | | Number of | Treatment |
|---|---|---|---|
| | | Subjects | Success (%) |
| Tazarotene | Composition A Lotion, | 402 | 25.5 |
| Products | 0.045% tazarotene | 397 | 29.6 |
| | Tazaorac Cream, 0.1% | 218 | 18 |
| | tazarotene | 206 | 20 |
| | Fabior Foam, 0.1% tazarotene | 371 | 29 |
| | | 373 | 28 |
| Tretinoin | Altreno Lotion, 0.05% | 406 | 16.5 |
| Products | tretinoin | 413 | 19.8 |
| | Atralin Gel, 0.05% tretinoin | 375 | 21 |
| | | 299 | 23 |
| Adapalene | Adapalene Gel, 0.3% | 258 | 21 |
| Products | Adapalene Gel, 0.1% | 261 | 16 |
| | Adapalene Lotion, 0.1% | 533 | 26.3 |
| | | 535 | 24.1 |
| | Epiduo Gel, 0.1% adapalene/ | 149 | 21.5 |
| | 2.5% benzoyl peroxide | 415 | 30.1 |
| | 0.1% Adapalene in Epiduo | 148 | 12.2 |
| | vehicle | 420 | 19.8 |

Surprisingly, Composition A Lotion achieved at least similar or higher success rate than other products that contain at least twice the concentration of the active ingredient.

What is claimed is:

1. A method of treating acne vulgaris in a subject with acne vulgaris and oily skin, the method comprising topically applying a pharmaceutical composition to an affected area of a body of a subject suffering from acne vulgaris and oily skin; wherein the composition comprises:
tazarotene or a pharmaceutically acceptable tazarotenic acid salt present in the composition at a concentration from about 0.01 to 0.049 percent by weight of the composition; and
a dermatologically acceptable oil-in-water emulsion vehicle;
wherein said composition is a lotion.

2. The method of claim 1, the pharmaceutical composition comprising tazarotene at a concentration from about 0.03 to 0.049 percent by weight of the composition.

3. The method of claim 1 or claim 2, the composition comprising tazarotene at a concentration of about 0.045 percent by weight of the composition.

4. The method of any one of claims 1 to 3, wherein said emulsion comprises:
an aqueous phase comprising water, a carbomer homopolymer, and a polymeric emulsifier; and
an oil phase comprising at least one member selected from the group consisting of a dicarboxylic acid ester and a mineral oil.

5. The method of claim 4, wherein the oil phase of the emulsion comprises diethyl sebacate.

6. The method of any one of claims 1 to 5, wherein the oil phase of the emulsion comprises diethyl sebacate and mineral oil.

7. The method of any one of claims 1 to 6, wherein the oil phase of the emulsion consists of diethyl sebacate and mineral oil.

8. The method of any one of claims 1 to 7, wherein said applying is carried out once per day for at least twelve weeks.

9. The method of any one of claims 1 to 8, wherein said applying is carried out once per day for twelve weeks.

10. The method of any one of claims 1 to 9, wherein the subject has dark skin.

11. The method of any one of claims 1 to 10, wherein the subject has black skin.

12. The method of any one of claims 1 to 11, wherein the subject is Black.

13. The method of any one of claims 1 to 12, wherein said applying is carried out once per day for at least twelve weeks.

14. The method of any one of claims 1 to 13, wherein the pharmaceutical composition consists essentially of:
0.045 weight % tazarotene;
diethyl sebacate;
light mineral oil;
sorbitan monooleate;
sorbitol solution, 70%;
methylparaben;
propylparaben;
edetate disodium dihydrate;
carbomer polymer type B;
carbomer homopolymer type A;
sodium hydroxide;
purified water.

15. The method of any one of claims 1 to 14, wherein the pharmaceutical composition consists essentially of:
0.045 weight % tazarotene;
2.5-3.5 weight % diethyl sebacate;
7.5-8.5 weight % light mineral oil;
0.05-0.2 weight % sorbitan monooleate;
10-11 weight % sorbitol solution, 70%;
0.1-0.2 weight % methylparaben;
0.02-0.04 weight % propylparaben;
0.03-0.06 weight % edetate disodium dihydrate;
0.3-0.5 weight % carbomer polymer type B;
0.5-0.7 weight % carbomer homopolymer type A;
sodium hydroxide, q.s. to pH 5.5±0.5; and
purified water, q.s. to 100 weight %.

16. A method of treating acne vulgaris in a subject with acne vulgaris and oily skin, the method comprising topically applying a pharmaceutical composition to an affected area of the body of the subject suffering from acne vulgaris and oily skin; wherein the composition consists essentially of:
0.045 weight % tazarotene;
2.97 weight % diethyl sebacate;
8.03 weight % light mineral oil;
0.1 weight % sorbitan monooleate;
10.7 weight % sorbitol solution, 70%;
0.17 weight % methylparaben;
0.03 weight % propylparaben;
0.05 weight % edetate disodium dihydrate;
0.4 weight % carbomer polymer type B;
0.6 weight % carbomer homopolymer type A;
sodium hydroxide, q.s. to pH 5.5±0.5; and
purified water, q.s. to 100 weight %.

17. A method for reducing skin oiliness in a subject with acne vulgaris and oily skin, the method comprising topically applying a pharmaceutical composition to an affected area of a body of a subject suffering from acne vulgaris and oily skin; wherein the composition; wherein the composition consists essentially of:
0.045 weight % tazarotene;
2.97 weight % diethyl sebacate;
8.03 weight % light mineral oil;
0.1 weight % sorbitan monooleate;
10.7 weight % sorbitol solution, 70%;
0.17 weight % methylparaben;
0.03 weight % propylparaben;
0.05 weight % edetate disodium dihydrate;
0.4 weight % carbomer polymer type B;
0.6 weight % carbomer homopolymer type A;
sodium hydroxide, q.s. to pH 5.5±0.5; and
purified water, q.s. to 100 weight %.

18. A method of treating truncal acne in a subject in need thereof, the method comprising topically applying a pharmaceutical composition to an affected area of the trunk of the subject suffering from truncal acne; wherein the composition comprises:
tazarotene or a pharmaceutically acceptable tazarotenic acid salt present in the composition at a concentration from about 0.01 to 0.049 percent by weight of the composition; and
a dermatologically acceptable oil-in-water emulsion vehicle;
wherein said composition is a lotion.

19. The method of claim 18, comprising tazarotene at a concentration from about 0.03 to 0.049 percent by weight of the composition.

20. The method of claim 18 or 19, the pharmaceutical composition comprising tazarotene at a concentration of about 0.045 percent by weight of the composition.

21. The method of any one of claims 18 to 20, wherein said emulsion comprises:
an aqueous phase comprising water, a carbomer homopolymer, and a polymeric emulsifier; and
an oil phase comprising at least one member selected from the group consisting of a dicarboxylic acid ester and a mineral oil.

22. The method of claim 21, wherein the oil phase of the emulsion comprises diethyl sebacate.

23. The method of any one of claims 18 to 22, wherein the oil phase of the emulsion comprises diethyl sebacate and mineral oil.

24. The method of any one of claims 18 to 23, wherein the oil phase of the emulsion consists of diethyl sebacate and mineral oil.

25. The method of any one of claims 18 to 24, wherein said applying is carried out once per day for at least twelve weeks.

26. The method of any one of claims 18 to 25, wherein said applying is carried out once per day for twelve weeks.

27. The method of any one of claims 18 to 26, wherein said applying is carried out once per day for at least twelve weeks.

28. The method of any one of claims 18 to 27, wherein the pharmaceutical composition consists essentially of:
0.045 weight % tazarotene;
diethyl sebacate;
light mineral oil;
sorbitan monooleate;
sorbitol solution, 70%;
methylparaben;
propylparaben;
edetate disodium dihydrate;
carbomer polymer type B;
carbomer homopolymer type A;
sodium hydroxide;
purified water.

29. The method of any one of claims 18 to 28, wherein the pharmaceutical composition consists essentially of:
0.045 weight % tazarotene;
2.5-3.5 weight % diethyl sebacate;
7.5-8.5 weight % light mineral oil;
0.05-0.2 weight % sorbitan monooleate;
10-11 weight % sorbitol solution, 70%;
0.1-0.2 weight % methylparaben;
0.02-0.04 weight % propylparaben;
0.03-0.06 weight % edetate disodium dihydrate;
0.3-0.5 weight % carbomer polymer type B;
0.5-0.7 weight % carbomer homopolymer type A;
sodium hydroxide, q.s. to pH 5.5±0.5; and
purified water, q.s. to 100 weight %.

30. A method of treating truncal acne in a subject in need thereof, the method comprising topically applying a pharmaceutical composition to an affected area of the trunk of the subject; wherein the composition consists essentially of:
0.045 weight % tazarotene;
2.97 weight % diethyl sebacate;
8.03 weight % light mineral oil;
0.1 weight % sorbitan monooleate;
10.7 weight % sorbitol solution, 70%;
0.17 weight % methylparaben;
0.03 weight % propylparaben;
0.05 weight % edetate disodium dihydrate;
0.4 weight % carbomer polymer type B;
0.6 weight % carbomer homopolymer type A;
sodium hydroxide, q.s. to pH 5.5±0.5; and
purified water, q.s. to 100 weight %.
